(19) United States
(12) Patent Application Publication
(10) Pub. No.: US 2023/0346893 A1
(43) Pub. Date: Nov. 2, 2023

(54) Methods and Compositions for Preventing or Minimizing Epithelial-Mesenchymal Transition

(71) Applicant: Innovon Pharmaceuticals Inc., Montrael (CA)

(72) Inventors: PIERRE LAURIN, Ville Mont-Royal (CA); LYNE GAGNON, Laval (CA)

(21) Appl. No.: 18/132,517

(22) Filed: Apr. 10, 2023

Related U.S. Application Data
(63) Continuation of application No. 16/472,627, filed on Jun. 21, 2019.
(60) Provisional application No. 62/462,530, filed on Feb. 23, 2017.
(60) Provisional application No. 62/437,123, filed on Dec. 21, 2016.

(51) Int. Cl. A61K 38/38; A61P 35/00; A61K 31/23

(52) U.S. Cl. CPC A61K 38/385; A61P 35/00; A61K 31/23

(57) ABSTRACT

Compositions, including albumin compositions, which inhibit or reduce epithelial-mesenchymal transition (EMT) are described. In embodiments, such compositions comprise albumin and either i) no pro-EMT agent or a low concentration of pro-EMT agent; ii) a content of pro-EMT agent and anti-EMT agent in a ratio of pro-EMT agent: anti-EMT agent that is from 7:3 to 0:10; or iii) both. The pro-EMT agent is octanoic acid, octanoate salt or a combination thereof. The anti-EMT agent is a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof. Use of such albumin compositions for various therapeutic applications based on albumin, and more particularly for the treatment of diseases or conditions in which EMT should be prevented or minimized. These albumin compositions can advantageously be used in combination with known active ingredients. Also, described are composition of an anti-EMT agent for use in the treatment of various therapeutic indications, and more particularly for the treatment of diseases or conditions where EMT should be prevented or minimized.

### FIELD OF INVENTION

The present invention is concerned with methods for preventing or minimizing epithelial-mesenchymal transition (EMT), and the corresponding use for the preparation of a medicament for preventing or minimizing epithelial-mesenchymal transition (EMT). More particularly, the present invention relates to albumin preparations, and uses thereof in various medicinal applications. The albumin preparation comprises no pro-EMT agent or a low concentration of pro-EMT agent; or an anti-EMT agent, wherein the ratio pro-EMT agent: anti-EMT agent is from 7:3 to 0:10. The anti-EMT agent is a fatty acid comprising an alkyl chain of 9 to 14 carbons, or a salt thereof, or a monoglyceride thereof, or a diglyceride thereof, or a triglyceride thereof, or a combination thereof. The pro-EMT agent is octanoic acid (C8 fatty acid) or octanoate salt (salt of C8 fatty acid).

The present invention also relates to methods and uses of an anti-EMT agent in the treatment of various medical conditions.

### BACKGROUND OF INVENTION

### Albumin

Human serum albumin (HSA) is the most abundant protein present in human blood plasma (concentration of 35-50 g/L). HSA is soluble in plasma and exists as a protein monomer (MW=66,500). It is produced in the liver and performs multiple functions, most of which involve the transport of small molecules. HSA preferentially binds hydrophobic carboxylic acids. Molecules that bind to and are transported by HSA include free fatty acids, hormones such as thyroid hormone, unconjugated bilirubin and a variety of drugs. As such, HSA can extend the half-life of drugs by binding them resulting in subsequent retardation of catabolic processes. Due to the relatively high concentration of HSA in the systemic circulation, it maintains the colloid osmotic (oncotic) pressure within the circulatory system. Oncotic pressure arises primarily from the inability of the large HSA molecule to escape through blood vessel walls.

### Heat Treatment of Albumin

As such, HSA is widely used for the treatment of hypovolemia. Hypovolemia refers to the state that results upon a significant decrease in blood volume arising from a loss of blood plasma. Several causes can give rise to hypovolemia which include trauma, burns or surgery. Conventional HSA is prepared by the low temperature ethanol fractionation of blood plasma: the Cohn process (developed during World War II) and variants thereof. However, the purified HSA may contain viral pathogens which necessitates a process of thermal treatment at 60° C. for 10 hours, or heat pasteurization. Albumin is prone to denaturation and subsequent formation of insoluble (non-functional) aggregates during thermal treatment (e.g. sterilization). Therefore, in order to minimize denaturation of HSA due to thermal treatment, commercial HSA formulations are prepared by addition of the medium-chain fatty acid, octanoic acid, and N-acetyl tryptophan prior to pasteurization. It has been shown that sodium octanoate protects monomeric HSA against heat while N-acetyl tryptophan protects against oxidative stress; M. Anraku et al., Biochim. Biophys. ACTA 1702(1), 9-17 (2004). The stabilization of albumin using medium-chain and long-chain fatty acids is also described in Shrake et al. Biopolymers 81(4), 235-248 (2006), U.S. patent application 2015/0165000 and U.S. Pat. No. 7,351,800. Notably because of its ready availability and its greater solubility relative to long-chain fatty acids, sodium octanoate constitutes the main stabilizer currently used as the stabilizer for commercial albumin preparations. Further, after decades of use, it has been established that sodium octanoate and N-acetyl tryptophan stabilized HSA is relatively safe for use in subjects with a healthy liver capable of metabolizing sodium octanoate and N-acetyl tryptophan.

### Therapeutic Uses of Albumin Preparations

Preparations containing albumin are often provided therapeutically to humans and animals. For example, preparations containing albumin are commonly administered to humans for one or more of the following indications: hypovolemia, with or without shock; hypoalbuminemia, which may result from inadequate production of albumin (due to malnutrition, burns, major injury, congenital analbuminemia, liver disease, infection, malignancy, or endocrine disorders), excessive catabolism (due to burns, major injury, pancreatitis, thyrotoxicosis, pemphigus, or nephrosis), loss of albumin from the body (due to hemorrhage, excessive renal excretion, burn exudates, exudative enteropathy, or exfoliative dermatoses) and/or redistribution of albumin within the body (due to major surgery, cirrhosis with ascites, or various inflammatory conditions); prior to or during cardiopulmonary bypass surgery; and for the treatment of burns or cirrhosis.

A number of different preparations containing albumin (typically at a concentration of 5 or 25%) for therapeutic use are or have been commercially available, including, for example:
Albuminar®-25 (Centeon/Aventis Behring) contains a sterile aqueous solution of 25% of albumin that is obtained from large pools of adult human venous plasma by low temperature controlled fractionation according to the Cohn process. It is stabilized with 0.02 M sodium N-acetyl tryptophan and 0.02 M sodium caprylate and pasteurized at 60° C. for 10 hours.
Buminate® (Baxter Laboratories) is a sterile aqueous solution of albumin prepared from human venous plasma using the Cohn cold ethanol fractionation process, adjusted to physiological pH with sodium bicarbonate and/or sodium hydroxide and stabilized with N-acetyl tryptophan (0.016M) and sodium caprylate (0.016M). Buminate® 5% contains 5% (weight/volume) of albumin and is stabilized with N-acetyl tryptophan (0.004 M) and sodium caprylate (0.004M). Buminate® 25% contains 25% (weight/volume) of albumin and is stabilized with N-acetyl tryptophan (0.02 M) and sodium caprylate (0.02 M). The sodium ion content is 145±15 mEq/L. The solutions contain no preservative and none of the coagulation factors found in fresh whole blood or plasma.
Plasbumin®-25 (Grifols) is a sterile solution of albumin 25% (weight/volume) in an aqueous diluent. The preparation is stabilized with 0.02 M sodium caprylate and 0.02 M N-acetyl tryptophan and buffered with sodium carbonate. The aluminum content of the product is not more than 200 μg/L. The approximate sodium ion content of the product is 145 mEq/L.
Albutein® 25% (Grifols) is a sterile aqueous solution containing 25% (weight/volume) of human albumin that is obtained by a cold alcohol fractionation method from pooled human plasma obtained from venous blood. The product is stabilized with 0.08 millimole sodium caprylate and 0.08 millimole sodium N-acetyl tryptophan per gram of albumin. The 0.08 millimole sodium caprylate per gram of albumin corresponds to 0.02 M sodium caprylate in a solution of 25% albumin. Albutein® 25% solution contains 130-160 milliequivalents of sodium ion per liter and has a pH of 6.9±0.5. The product contains no preservatives.
Albumarc® (American Red Cross) is a sterile aqueous solution of 5% or 25% (weight/volume) albumin, prepared from human venous plasma using the Cohn cold ethanol fractionation process, adjusted to physiological pH with sodium bicarbonate and/or sodium hydroxide and stabilized with sodium caprylate and sodium N-acetyl tryptophan.
Recombumin Prime® (Albumedix/Novozymes) is a recombinant human albumin (rAlbumin) manufactured to ICH Q7 standards. The source of Recombunin® is recombinant Saccharomyces cerevisiae (baker's yeast) fermentation and it is manufactured without the use of animal or human derived materials. Recombumin Prime® is sold in a 50 ml Type II glass vial containing a 20% (w/v) protein solution. It is stabilized with 32 mM sodium caprylate, 145 mM of sodium chloride, and 15 mg/L of Polysorbate 80.
Recombumin 20% Alpha® (Albumedix/Novozymes) is a recombinant human albumin (rAlbumin) manufactured to ICH Q7 standards. The source of Recombunin is recombinant Saccharomyces cerevisiae (baker's yeast) fermentation and it is manufactured without the use of animal or human derived materials. Recombumin Alpha@ is sold in a 50 ml Type II glass vial containing a 20% (w/v) protein solution. It is stabilized with 16 mM sodium caprylate, and 145 mM of sodium chloride.
Recombumin 10% Alpha® (Albumedix/Novozymes) is a recombinant human albumin (rAlbumin) manufactured to ICH Q7 standards. The source of Recombunin® is recombinant Saccharomyces cerevisiae (baker's yeast) fermentation and it is manufactured without the use of animal or human derived materials Recombumin Alpha® is sold in a 50 ml Type II glass vial containing a 10% (w/v) protein solution. It is stabilized with 8 mM sodium caprylate, and 145 mM of sodium chloride.
Albagen® 5% (New Century Pharmaceuticals) is a recombinant human albumin (rAlbumin) manufactured with Pichia pastoris. Derived from the prototypical human serum albumin sequence with a single deletion at the N-terminus (Asp). It has all of the biological properties of Human Serum Albumin with reduced high affinity site for nickel and copper. Sterile 5% solution in PBS containing 4 mM sodium caprylate and 4 mM N-acetyl tryptophan.
Kedbumin® (Kedrion Biopharma) is a sterile, aqueous solution for single dose intravenous administration containing 25% albumin that is prepared by cold ethanol fractionation from pooled human plasma. The product is stabilized by the addition of 0.08 mmol sodium caprylate and 0.08 mmol sodium N-acetyltryptophan per gram of albumin. The 0.08 mmol sodium caprylate per gram of albumin corresponds to 0.02 M sodium caprylate in a solution of 25% albumin. Additionally, each liter of material contains 130-160 mEq of sodium ion and ≤200 μg of aluminum. The product contains no preservatives.

Thus, commercial human albumin preparations are typically stabilized using octanoic/caprylic acid (or sodium octanoate/caprylate).

### Albumin as a Delivery Vehicle for Drugs

HSA has been considered as a potential delivery vehicle for lipophilic anticancer drugs. For example, Abraxane™ (nanoparticle albumin-bound paclitaxel) has been approved in the US for metastatic breast cancer (2005), non-small cell lung cancer (2012) and metastatic pancreatic cancer (2013). In this case, lipophilic paclitaxel is formulated as albumin-bound nanoparticles (average size: 130 nM) for use as an injectable suspension upon reconstitution with 0.9% sodium chloride; the HSA being previously treated with sodium octanoate (sodium caprylate) and N-acetyl tryptophan (sodium N-acetyl tryptophanate). The potential use of HSA as a delivery vehicle for other poorly water soluble cancer drugs has been documented in the literature. For example, Q. Li et al., Int. J. Nanomedicine 6, 397-405 (2011) report that folate conjugated HSA-10-hydroxycamptothecin-loaded nanoparticles demonstrated sustained-release behavior and an effective delivery system for drug up-take by cancer cells. A later publication in the same journal demonstrated that 10-hydroxycamptothecin-loaded glycyrrhizic acid-conjugated albumin nanoparticles are a promising vehicle for hepatocellular carcinoma targeting therapy; Y. Zu et al., Int. J. Nanomedicine 8, 1207-1222 (2013). Albumin nanoparticles have also been studied as a delivery vehicle for the cancer drug doxorubicin, for example, by S. Abbasi et al. (J. Drug Delivery 2012: Article ID 686108, 8 pages), who report that modification of HSA nanoparticles by addition of a coating of polyethylenimine gave a cationic albumin nanoparticle that improved the therapeutic index of doxorubicin against MCF-7 breast cancer cells. It was observed that the net positive charge nanoparticles improved penetration into the cancer cells (compared to HSA) thereby yielding a more potent cytotoxic effect on the cancer cells over a longer time duration.

There is a continued need for stable and safe albumin preparations.

The present description refers to a number of documents, the content of which is herein incorporated by reference in their entirety.

### SUMMARY OF THE INVENTION

The present invention relates to the following items:
Item 1: A method of treating a disease or condition with a composition comprising albumin while preventing or minimizing epithelial-mesenchymal transition (EMT), said method comprising administering to a subject in need thereof an effective amount of said composition, wherein said composition contains no octanoic acid and/or octanoate salt or a reduced amount of octanoic acid and/or octanoate salt, wherein said reduced amount is less than 0.08 millimole per gram of albumin.
Item 2: The method of item 1, wherein the reduced amount of octanoic acid and/or octanoate salt is less than 0.05 millimole per gram of albumin.
Item 3: The method of item 1, wherein the reduced amount of octanoic acid and/or octanoate salt is less than 0.005 millimole per gram of albumin.
Item 4: The method of item 1, wherein the reduced amount of octanoic acid and/or octanoate salt is less than 0.0005 millimole per gram of albumin.
Item 5: The method of item 1, wherein said composition is substantially free of octanoic acid or octanoate salt.
Item 6: The method of item 1, wherein said composition is free of octanoic acid or octanoate salt.
Item 7: The method of item 1, wherein said composition contains an amount of fatty acid and/or a salt thereof that is less or equal to 0.08 millimole per gram of albumin, wherein at least 30% of said amount of fatty acid and/or salt thereof is C9-C14 fatty acid and/or salt thereof.
Item 8: The method according to item 7, wherein at least 50% of said fatty acid and/or salt thereof is a C9-C14 fatty acid and/or salt thereof.
Item 9: The method according to item 7, wherein at least 70% of said fatty acid and/or salt thereof is a C9-C14 fatty acid and/or salt thereof.
Item 10: The method according to item 7, wherein at least 90% of said fatty acid and/or salt thereof is a C9-C14 fatty acid and/or salt thereof.
Item 11: The method according to item 7, wherein at least 95% of said fatty acid and/or salt thereof is a C9-C14 fatty acid and/or salt thereof.
Item 12: The method according to item 7, wherein 100% of said fatty acid and/or salt thereof is a C9-C14 fatty acid and/or salt thereof.
Item 13: The method of any one of items 1 to 12, wherein said composition is an aqueous albumin preparation comprising from about 1% to about 40% (w/v) of albumin.
Item 14: The method of item 13, wherein said composition is an aqueous albumin preparation comprising from about 5% to about 25% (w/v) of albumin.
Item 15: The method of item 13, wherein said composition is an aqueous albumin preparation comprising from about 20% to about 25% (w/v) of albumin.
Item 16: The method according to any one of items 7 to 12, wherein said C9-C14 fatty acid and/or salt thereof is a C9-C12 fatty acid and/or salt thereof.
Item 17: The method according to item 16, wherein said C9-C12 fatty acid and/or salt thereof is decanoic acid or a salt of decanoic acid.
Item 18: The method according to any one of items 7 to 12 and 16-17, wherein the salt is a salt of sodium, potassium, lithium, ammonium, calcium, magnesium, manganese, zinc, iron, or copper.
Item 19: The method according to item 18, wherein the salt is a salt of sodium or potassium.
Item 20: The method according to item 18, wherein the salt is a salt of sodium.
Item 21: The method according to any one of items 1 to 20, wherein said albumin is human serum albumin (HSA).
Item 22: The method according to any one of items 1 to 21, wherein said composition is in a liquid form, and further comprises one or more pharmaceutically acceptable osmolality regulators.
Item 23: The method according to item 22, wherein said one or more osmolality regulators comprise a sodium salt, a potassium salt, or both.
Item 24: The method according to any one of items 1 to 23, wherein said composition is in a liquid form, and further comprises at least one pharmaceutically acceptable antioxidant or stabilizer.
Item 25: The method according to item 24, wherein said at least one pharmaceutically acceptable antioxidant or stabilizer is an antioxidant amino acid or a derivative thereof.
Item 26: The method according to item 24, wherein said at least one pharmaceutically acceptable antioxidant or stabilizer is an N-acyl methionine or N-acyl tryptophanate.
Item 27: The method according to any one of items 1 to 21, wherein the composition is in the form of nanoparticles.
Item 28: The method according to any one of items 1 to 27, wherein the composition further comprises a pharmaceutically active agent.
Item 29: The method according to item 27, wherein the composition further comprises a pharmaceutically active agent; and wherein said pharmaceutically active agent is encapsulated within the nanoparticles.
Item 30: The method according to item 28 or 29, wherein said pharmaceutically active agent is an anticancer drug.
Item 31: The method according to item 30, wherein said anticancer drug is a taxane, camptothecin, irinotecan, gemcitabine, cytoxan or doxorubicin.
Item 32: The method according to item 31, wherein said taxane is paclitaxel or docetaxel.
Item 33: The method according to any one of items 1 to 30, wherein said disease or condition is hemorrhage, hypovolemic shock, burns, acute liver failure, hypoalbuminemia, adult respiratory distress syndrome, cirrhosis, neonatal hemolytic disease, cardiopulmonary bypass surgery, nephrosis or cancer.
Item 34: The method according to any one of items 30 to 32, wherein said disease or condition is cancer.
Item 35: Use of a composition comprising albumin for treating a disease or condition while preventing or minimizing epithelial-mesenchymal transition (EMT), wherein said composition contains no octanoic acid and/or octanoate salt or a reduced amount of octanoic acid and/or octanoate salt, wherein said reduced amount is less than 0.08 millimole per gram of albumin.
Item 36: Use of item 35, wherein said composition contains an amount of fatty acid and/or a salt thereof, wherein said amount is equal or less than 0.08 millimole per gram of albumin, wherein at least 30% of said amount of fatty acid and/or salt thereof is C9-C14 fatty acid and/or salt thereof.
Item 37: Use of a composition comprising albumin for the manufacture of a medicament for treating a disease or condition while preventing or minimizing epithelial-mesenchymal transition (EMT), wherein said composition contains no octanoic acid and/or octanoate salt or a reduced amount of octanoic acid and/or octanoate salt, wherein said reduced amount is less than 0.08 millimole per gram of albumin.
Item 38: Use of item 37, wherein said composition contains an amount of fatty acid and/or a salt thereof, wherein at least 30% of said amount of fatty acid and/or salt thereof is C9-C14 fatty acid and/or salt thereof.
Item 39: A composition comprising albumin for use in treating a disease or condition while preventing or minimizing epithelial-mesenchymal transition (EMT), wherein said composition contains no octanoic acid and/or octanoate salt or a reduced amount of octanoic acid and/or octanoate salt, wherein said reduced amount is less than 0.08 millimole per gram of albumin.
Item 40: The composition of item 39, containing an amount of fatty acid and/or a salt thereof that is equal or less than 0.08 millimole per gram of albumin, wherein at least 30% of said amount of fatty acid and/or salt thereof is C9-C14 fatty acid and/or salt thereof.
Item 41: A composition comprising albumin, and
   an amount of octanoic acid and/or octanoate salt that is less than 0.08 millimole per gram of albumin; and/or
   an amount of fatty acid and/or a salt thereof wherein at least 30% of said amount of fatty acid and/or salt thereof is C9-C14 fatty acid and/or salt thereof.
Item 42: The composition of item 41, wherein the amount of octanoic acid and/or octanoate salt is less than 0.05 millimole per gram of albumin.
Item 43: The composition of item 42, wherein the amount of octanoic acid and/or octanoate salt is less than 0.005 millimole per gram of albumin.
Item 44: The composition of item 43, wherein the amount of octanoic acid and/or octanoate salt is less than 0.0005 millimole per gram of albumin.
Item 45: The composition according to any one of items 41 to 44, wherein albumin is in a concentration of 1% to 40%.
Item 46: The composition according to item 45, wherein albumin is in a concentration of 5% to 25%.
Item 47: The composition according to any one of items 41 to 46, further comprising a pharmaceutically active agent.
Item 48: The composition according to item 47, wherein the pharmaceutically active agent is an anticancer drug.
Item 49: The composition according to item 48, wherein said anticancer drug is a taxane, camptothecin, irinotecan, gemcitabine, cytoxan or doxorubicin.
Item 50: The composition according to item 49, wherein said taxane is paclitaxel or docetaxel.
Item 51: The composition of any one of items 48 to 50, wherein the composition is substantially free of octanoic acid and/or octanoate salt.
Item 52: The composition according to any of items 41 to 51, for use in treating a disease or condition while preventing or minimizing epithelial-mesenchymal transition (EMT).
Item 53: The composition according to item 52, wherein said disease or condition is hemorrhage, hypovolemic shock, burns, acute liver failure, hypoalbuminemia, adult respiratory distress syndrome, cirrhosis, neonatal hemolytic disease, cardiopulmonary bypass surgery, or nephrosis.
Item 54: The composition according to any of items 48 to 50, for use in treating a disease or condition while preventing or minimizing epithelial-mesenchymal transition (EMT), wherein said disease or condition is cancer.
Item 55: A method of preventing, minimizing or reducing epithelial-mesenchymal transition (EMT) in a subject in need thereof, said method comprising administering to the subject a composition comprising an effective amount of C9-C14 fatty acid and/or salt thereof, and/or triglyceride of C9-C14 fatty acid.
Item 56: The method according to item 55, wherein said C9-C14 fatty acid and/or salt thereof, and/or triglyceride of C9-C14 fatty acid is C9-C12 fatty acid and/or salt thereof, and/or triglyceride of C9-C12 fatty acid.
Item 57: The method according to item 55, wherein said C9-C14 fatty acid and/or salt thereof, and/or triglyceride of C9-C14 fatty acid is C10-C12 fatty acid and/or salt thereof, and/or triglyceride of C10-C12 fatty acid.
Item 58: The method according to item 55, wherein said C9-C14 fatty acid and/or salt thereof, and/or triglyceride of C9-C14 fatty acid is decanoate.
Item 59: The method according to item 55, wherein said C9-C14 fatty acid and/or salt thereof, and/or triglyceride of C9-C14 fatty acid is sodium decanoate.
Item 60: The method according to item 55, wherein said C9-C14 fatty acid and/or salt thereof, and/or triglyceride of C9-C14 fatty acid is decanoic acid.
Item 61: The method according to item 55, wherein said C9-C14 fatty acid and/or salt thereof, and/or triglyceride of C9-C14 fatty acid is glyceryl tridecanoate.
Item 62: The method according to any one of items 55 to 58, wherein said composition comprises a salt of fatty acid, wherein the salt is sodium, potassium, lithium, ammonium, calcium, magnesium, manganese, zinc, iron, or copper.
Item 63: The method according to item 62, wherein said salt is a salt of sodium or potassium
Item 64: The method according to item 62, wherein said salt is a salt of sodium.
Item 65: The method according to any one of items 55 to 64, wherein said an effective amount is between 5 mg/kg to 300 mg/kg per dose for oral or topical administration.
Item 66: The method according to any one of items 65 wherein said an effective amount is between 10 mg/kg to 100 mg/kg per dose for oral or topical administration.
Item 67: The method according to any one of items 55 to 63, wherein said an effective amount is between 0.5 mg/kg to 100 mg/kg per dose for intravenous, intraperitoneal, rectal, intramuscular or subcutaneous administration.
Item 68: The method according to any one of items 67, wherein said an effective amount is between 0.5 mg/kg to 4 mg/kg per dose for intravenous, intraperitoneal, rectal, intramuscular or subcutaneous administration.
Item 69: The method according to any one of items 55 to 64, wherein said composition is administered topically.
Item 70: The method according to item 69, wherein said subject suffers from burns or scars.
Item 71: The method according to item 55 to 64 and 67-68, wherein said disease or condition is hemorrhage, hypovolemic shock, burns, acute liver failure, hypoalbuminemia, adult respiratory distress syndrome, cirrhosis, neonatal hemolytic disease, cardiopulmonary bypass surgery, or nephrosis.
Item 72: Use of C9-C14 fatty acid and/or salt thereof, and/or triglyceride of C9-C14 fatty acid, for preventing, minimizing or reducing epithelial-mesenchymal transition (EMT) in a subject.
Item 73: Use of C9-C14 fatty acid and/or salt thereof, and/or triglyceride of C9-C14 fatty acid, for the preparation of a medicament for preventing, minimizing or reducing epithelial-mesenchymal transition (EMT) in a subject.
Item 74: The use according to item 72 or 73, wherein said method comprising administering to the subject an effective amount of decanoic acid and/or decanoate salt.
Item 75: The use according to item 72 or 73, wherein said method comprising administering to the subject an effective amount of glyceryl tridecanoate.
Item 76: A use of a composition of albumin for the preparation of a medicament for treating a disease or a condition in a subject in need of receiving a composition of albumin for the treatment of said disease or condition, wherein the composition of albumin inhibits or reduces epithelial-mesenchymal transition (EMT), and contains:
   no pro-EMT agent or a low concentration of pro-EMT agent; and/or
   a content of pro-EMT agent and anti-EMT agent in a ratio of pro-EMT agent: anti-EMT agent that is from 7:3 to 0:10;
      wherein:
the pro-EMT agent is octanoic acid, octanoate salt or a combination thereof;
the anti-EMT agent is a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof; and
   said low concentration of pro-EMT agent is less than 0.08 millimole per gram of albumin;
   with the proviso that, when the pro-EMT agent and the anti-EMT agent are fatty acids, they are separated fatty acids or part of a diglyceride or a triglyceride.
Item 77: The use of claim 76, wherein the low concentration of pro-EMT agent is 0.04 millimole per gram of albumin or less.
Item 78: The use of claim 76, wherein the low concentration of pro-EMT agent is 0.007% (w/w) or less.
Item 79: The use of claim 76, wherein the ratio of pro-EMT agent: anti-EMT agent is from 5:5 to 0:10.
Item 80: The use of any one of claims 76 to 79, wherein the anti-EMT agent is a C9-C12 fatty acid, a salt of C9-C12 fatty acid, a monoglyceride of C9-C12 fatty acid, a diglyceride of C9-C12 fatty acid, a triglyceride of C9-C12 fatty acid, or a combination thereof.
Item 81: The use of claim 80, wherein the anti-EMT agent is a C10-C12 fatty acid, a salt of C10-C12 fatty acid, a monoglyceride of C10-C12 fatty acid, a diglyceride of C10-C12 fatty acid, a triglyceride of C10-C12 fatty acid, or a combination thereof.
Item 82: The use of claim 81, wherein the anti-EMT agent is a C10 fatty acid, a salt of C10 fatty acid, or a combination thereof.
Item 83: The use of any one of claims 76 to 82, wherein said composition is an aqueous albumin preparation comprising from about 1% to about 40% (w/v) of albumin.
Item 84: The use of any one of claims 76 to 83, wherein the salt is a salt of sodium, potassium, lithium, ammonium, calcium, magnesium, manganese, zinc, iron, copper, or a combination thereof.
Item 85: The use of claim 84, wherein the salt is a salt of sodium.
Item 86: The use of any one of claims 76 to 85, wherein the composition further comprises a pharmaceutically active agent.
Item 87: The use of claim 86, wherein said active agent is an anticancer drug, and the medicament is for treating cancer.
Item 88: The use of claim 87, wherein the anticancer drug is a taxane.
Item 89: The use of claim 88, wherein taxane is paclitaxel or docetaxel.
Item 90: The use of any one of claims 76 to 86, wherein the composition of albumin is recommended by a physician for the treatment of said disease or condition.
Item 91: The use of any one of claims 76 to 90, wherein the medicament is for treating disease or a condition that is hemorrhage, hypovolemia, burn, acute liver failure, liver dysfunction, hypoalbuminemia, adult respiratory distress syndrome, cirrhosis, neonatal hemolytic disease, cardiopulmonary bypass surgery, nephrosis, cancer, hepatorenal syndrome, sepsis, organ perfusion, or organ reperfusion.
Item 92: A use of a composition for the preparation of a medicament for inhibiting or reducing epithelial-mesenchymal transition (EMT) in a subject, wherein the composition comprises:
   no pro-EMT agent or a low concentration of pro-EMT agent; and/or
   a content of pro-EMT agent and anti-EMT agent in a ratio of pro-EMT agent: anti-EMT agent that is from 7:3 to 0:10;
      wherein:
the pro-EMT agent is octanoic acid, octanoate salt or a combination thereof;
the anti-EMT agent is a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof; and
   said low concentration of pro-EMT agent is less than 0.02 M;
   with the proviso that, when the pro-EMT agent and the anti-EMT agent are fatty acids, they are separated fatty acids or part of a diglyceride or a triglyceride.
Item 93: The use of claim 92, wherein the low concentration of pro-EMT agent is 0.01 M or less.
Item 94: The use of claim 92, wherein the low concentration of pro-EMT agent is 0.001 M or less.
Item 95: The use of claim 92, wherein the ratio of pro-EMT agent: anti-EMT agent is from 5:5 to 0:10.
Item 96: The use of claim 95, wherein the ratio of pro-EMT agent: anti-EMT agent is about 0:10.
Item 97: The use of any one of claims 92 to 96, wherein the anti-EMT agent is a C9-C12 fatty acid, a salt of C9-C12 fatty acid, a monoglyceride of C9-C12 fatty acid, a diglyceride of C9-C12 fatty acid, a triglyceride of C9-C12 fatty acid, or a combination thereof.
Item 98: The use of claim 97, wherein the anti-EMT agent is a C10-C12 fatty acid, a salt of C10-C12 fatty acid, a monoglyceride of C10-C12 fatty acid, a diglyceride of C10-C12 fatty acid, a triglyceride of C10-C12 fatty acid, or a combination thereof.
Item 99: The use of claim 98, wherein the anti-EMT agent is a C10 fatty acid, a salt of C10 fatty acid, a monoglyceride of C10 fatty acid, a diglyceride of C10 fatty acid, a triglyceride of C10 fatty acid, or a combination thereof.
Item 100: The use of any one of claims 92 to 99, wherein the salt is a salt of sodium, potassium, lithium, ammonium, calcium, magnesium, manganese, zinc, iron, copper, or a combination thereof.
Item 101: The use of claim 100, wherein the salt is a salt of sodium.
Item 102: The use of any one of claims 92 to 101, wherein the composition further comprises a pharmaceutically active agent.
Item 103: The use of claim 102, wherein said active agent is an anticancer drug, and the medicament is for treating cancer.
Item 104: The use of claim 103, wherein the anticancer drug is a taxane.
Item 105: The use of claim 104, wherein taxane is paclitaxel or docetaxel.
Item 106: The use of any one of claims 92 to 102, wherein the medicament is for treating a disease or a condition that is hemorrhage, hypovolemia, burn, acute liver failure, liver dysfunction, hypoalbuminemia, adult respiratory distress syndrome, cirrhosis, neonatal hemolytic disease, cardiopulmonary bypass surgery, nephrosis, cancer, hepatorenal syndrome, sepsis, organ perfusion, organ reperfusion, scar formation, psoriasis or eczema.
Item 107: The use of any one of claims 92 to 102, wherein the total concentration of pro-EMT agent and anti-EMT agent is between 5 mg/kg to 300 mg/kg of the subject per dose for oral or topical administration.
Item 108: The use of claim 107, wherein said total concentration is between 10 mg/kg to 100 mg/kg per dose.
Item 109: The use of any one of claims 92 to 102, wherein said total concentration is between 0.5 mg/kg to 100 mg/kg of the subject per dose for intravenous, intraperitoneal, rectal, intramuscular or subcutaneous administration.
Item 110: The use of claim 109, wherein said total concentration is between 0.5 mg/kg to 4 mg/kg per dose.
Item 111: The use of any one of claims 92-102, 107 and 108, wherein said composition is administered topically, and the medicament is for treating burn, for preventing scar formation, or for treating scar, psoriasis or eczema.
Item 112: A method for treating a disease or a condition in a subject in need of receiving a composition of albumin for the treatment of said disease or condition, comprising the administration of a composition of albumin that inhibits or reduces the stimulation of epithelial-mesenchymal transition (EMT) and that contains:
   no pro-EMT agent or a low concentration of pro-EMT agent; or
   a content of pro-EMT agent and anti-EMT agent in a ratio of pro-EMT agent: anti-EMT agent that is from 7:3 to 0:10;
      wherein:
the pro-EMT agent is octanoic acid, octanoate salt or a combination thereof;
the anti-EMT agent is a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof; and
said low concentration of pro-EMT agent is less than 0.08 millimole per gram of albumin;
with the proviso that, when the pro-EMT agent and the anti-EMT agent are fatty acids, they are separated fatty acids or part of a diglyceride or a triglyceride.
Item 113: A method for inhibiting or reducing epithelial-mesenchymal transition (EMT) in a subject, comprising the administration of a composition comprising:
   no pro-EMT agent or a low concentration of pro-EMT agent; and/or
   a content of pro-EMT agent and anti-EMT agent in a ratio of pro-EMT agent: anti-EMT agent that is from 7:3 to 0:10;
      wherein:
the pro-EMT agent is octanoic acid, octanoate salt or a combination thereof;
the anti-EMT agent is a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof; and
said low concentration of pro-EMT agent is less than 0.02 M;
with the proviso that, when the pro-EMT agent and the anti-EMT agent are fatty acids, they are separated fatty acids or part of a diglyceride or a triglyceride.

### BRIEF DESCRIPTION OF THE DRAWINGS

In the Appended Drawings:

FIG. 1 shows HPLC chromatograms o: a solution of 40 mM sodium caprylate (upper panel), a solution of 40 mM sodium decanoate (middle panel) and a composition of 5% albumin after replacement of sodium octanoate by sodium decanoate as described in Example 1 (lower panel).

FIG. 2 shows the production of EMT marker (collagen) in the in vitro assay described in Example 4, for compositions comprising a ratio of sodium octanote: sodium decanoate of 100:0, 95:5, 70:30, 50:50, 30:70, 5:95 or 0:100, with albumin and without albumin.

FIG. 3 shows the amino acid sequence of a native HSA preprotein (SEQ ID NO:1, NCBI Reference Sequence: NP 000468.1, UniProtKB: P02768).

FIG. 4 shows the effect of sodium octanoate and sodium decanoate, in presence and in absence of albumin, on the collagen 1a1 mRNA expression (EMT marker) in TGF-β induced human microvascular endothelial cells (HMEC).

FIG. 5 shows the effect of sodium octanoate and sodium decanoate, in presence and in absence of albumin, on the collagen 1a1 mRNA expression (EMT marker) in TGF-β induced human hepatocellular carcinoma cell line HepG2.

FIG. 6 shows the effect of sodium octanoate and sodium decanoate, in presence and in absence of albumin, on the collagen 1a1 mRNA expression (EMT marker) in TGF-β induced human lung epithelial cell A549.

FIG. 7 presents pictures of a scratch assay using mitomycin-treated EGF-induced PC-3 cell, showing the effect of sodium octanoate and sodium decanoate, in presence and in absence of albumin, on the migration of the cells.

### DETAILED DESCRIPTION OF THE INVENTION

The present invention relates to compositions of albumin comprising:
no fatty acid or salt thereof, or
no pro-EMT agent, or
a low concentration of pro-EMT agent, and/or
a ratio of pro-EMT agent: anti-EMT agent that is from 7:3 to 0:10,
   wherein said pro-EMT agent is octanoic acid, octanoate salt, or a combination thereof; and wherein said anti-EMT agent is a fatty acid having an alkyl chain of 9, 10, 11, 12, 13 or 14 carbons (i.e. C9-C14 fatty acid), a salt of C9-C14 fatty acid, a monoglyceride of C9-C14 fatty acid, a diglyceride of C9-C14 fatty acid, a triglyceride of C9-C14 fatty acid, or a combination thereof.

The present invention is based on the discovery that octanoic acid and octanoate salt are pro-EMT, i.e. octanoic acid and octanoate salt stimulate epithelial-mesenchymal transition (EMT). The stimulation of EMT is detrimental in many medical conditions where physicians are used to prescribe the administration of albumin preparations, such as severe burns since EMT stimulation contributes to excessive scarring. Other therapeutic indications where the stimulation of EMT is detrimental, are described herein below.

The inventors have also discovered that other fatty acids, salts thereof and triglycerides thereof are anti-EMT. Said anti-EMT agent is a C9 fatty acid such as nonanoic acid or pelargonic acid, a salt of C9 fatty acid such as nonanoic salt, a monoglyceride of C9 fatty acid, a diglyceride of C9 fatty acid, a triglyceride of C9 fatty acid such as triglyceride of octanoic acid or glyceryl trinonanoate, a C10 fatty acid such as decanoic acid or capric acid, a salt of C10 fatty acid such as decanoate salt or caprate salt, a monoglyceride of C10 fatty acid, a diglyceride of C10 fatty acid, a triglyceride of C10 fatty acid such as triglyceride of decanoic acid or glyceryl tridecanoate or glyceryl tricaprin), a C11 fatty acid such as undecanoic acid or undecylic acid, a salt of C11 fatty acid such as undecanoate salt, a monoglyceride of C11 fatty acid, a diglyceride of C11 fatty acid, a triglyceride of C11 fatty acid such as triglyceride of undecanoic acid or glyceryl triundecanoate, a C12 fatty acid such as dodecanoic acid or lauric acid, a salt of C12 fatty acid such as dodecanoate salt, a monoglyceride of C12 fatty acid, a diglyceride of C12 fatty acid, a triglyceride of C12 fatty acid such as triglyceride of dodecanoic acid or glyceryl trilaurin, a C13 fatty acid such as tridecanoic acid or tridecylic acid, a salt of C13 fatty acid such as tridecanoic salt, a monoglyceride of C13 fatty acid, a diglyceride of C13 fatty acid, a triglyceride of C13 fatty acid such as triglyceride of tridecanoic acid or glyceryl tridodecanoate, a C14 fatty acid such as tetradecanoic acid, a salt of C14 fatty acid such as tetradecanoic salt, a monoglyceride of C14 fatty acid, a diglyceride of C14 fatty acid, a triglyceride of C14 fatty acid such as triglyceride of tetradecanoic acid or glyceryl tritetradecanoate or glyceryl trimyristin, or a combination thereof. The effect of the triglycerides of C9, C10, C11, C12, C13 and C14 fatty acids have been tested and since the triglyceride is broken down into one molecule of glycerol and three fatty acids after its passage in the intestine, it is reasonably expected that the corresponding monoglyceride and diglyceride will provide the same efficacy once the amount of monoglyceride or diglyceride is adjusted so as to free the same amount of fatty acids than the corresponding triglyceride. The anti-EMT effect of the triglyceride, diglyceride and monoglyceride is conveyed by the activity of the C9-C14 fatty acid that is released in vivo

When the anti-EMT agent is present in the albumin preparation, its concentration is represented by a ratio of pro-EMT: anti-EMT agent that is from 7:3 to 0:10, including 7:3, 6:4, 5:5, 4:6; 3:7, 2:8, 1:9 and 0:10. In an embodiment, the content of pro-EMT agent and anti-EMT agent in a ratio of pro-EMT agent: anti-EMT agent that is from 7:3 to 0:10 is embodied by a triglyceride composed of two octanoic acids and one C9-C14 fatty acid (i.e. ratio of 2:1, as exemplified in Example 4), or one octanoic acid and two C9-C14 fatty acids (i.e. ratio of 1:2), or by a diglyceride composed of one octanoic acid and one C9-C14 fatty acid (i.e. ratio of 5:5). In an embodiment, the content of pro-EMT agent in the composition is not at a low concentration (a low concentration of pro-EMT agent is defined below), but a content of an anti-EMT agent is such that the ratio of pro-EMT agent: anti-EMT agent is from 7:3 to 0:10, preferably 2:1, also preferably 1:1, further preferably 1:2, and also preferably from 1:1 to 0:1. In another embodiment, the content of pro-EMT agent in the composition is absent or at a low concentration (a low concentration of pro-EMT agent is defined below), and a content of an anti-EMT agent is such that the ratio of pro-EMT agent: anti-EMT agent is from 7:3 to 0:10, preferably 2:1, 6:4, 1:1, 1:2, 3:7, 2:8, 1:9 or 0:10.

Said anti-EMT agent is preferably C9-C14 fatty acid, salt thereof, monoglyceride thereof, diglyceride thereof or triglyceride thereof. The anti-EMT agent is preferably C9 fatty acid, salt thereof, monoglyceride thereof, diglyceride thereof or triglyceride thereof. The anti-EMT agent is preferably C10 fatty acid, salt thereof, monoglyceride thereof, diglyceride thereof or triglyceride thereof. The anti-EMT agent is preferably C11 fatty acid, salt thereof, monoglyceride thereof, diglyceride thereof or triglyceride thereof. The anti-EMT agent is preferably C12 fatty acid, salt thereof, monoglyceride thereof, diglyceride thereof or triglyceride thereof. The anti-EMT agent is preferably C13 fatty acid, salt thereof, monoglyceride thereof, diglyceride thereof or triglyceride thereof. The anti-EMT agent is preferably C14 fatty acid, salt thereof, monoglyceride thereof, diglyceride thereof or triglyceride thereof. The anti-EMT agent is preferably C9-12 fatty acid, salt thereof, monoglyceride thereof, diglyceride thereof or triglyceride thereof. The anti-EMT agent is preferably C10-12 fatty acid, salt thereof, monoglyceride thereof, diglyceride thereof or triglyceride thereof. The anti-EMT agent is preferably C10 fatty acid, salt thereof, or triglyceride thereof. The anti-EMT agent is preferably C10 fatty acid, or salt thereof. The anti-EMT agent is preferably a salt of C10 fatty acid. The anti-EMT agent is preferably C11 fatty acid, salt thereof, or triglyceride thereof. The anti-EMT agent is preferably C11 fatty acid, or salt thereof. The anti-EMT agent is preferably a salt of C11 fatty acid. The anti-EMT agent is preferably C12 fatty acid, salt thereof, or triglyceride thereof. The anti-EMT agent is preferably C12 fatty acid, or salt thereof. The anti-EMT agent is preferably a salt of C12 fatty acid. The anti-EMT agent is preferably C9 fatty acid, salt thereof, or triglyceride thereof. The anti-EMT agent is preferably C9 fatty acid, or salt thereof. The anti-EMT agent is preferably a salt of C9 fatty acid. The anti-EMT agent is preferably C10 fatty acid, salt thereof, or triglyceride thereof. The anti-EMT agent is preferably C13 fatty acid, or salt thereof, or triglyceride thereof. The anti-EMT agent is preferably C13 fatty acid, or salt thereof. The anti-EMT agent is preferably a salt of C13 fatty acid. The anti-EMT agent is preferably C14 fatty acid, salt thereof, or triglyceride thereof. The anti-EMT agent is preferably C14 fatty acid, or salt thereof. The anti-EMT agent is preferably a salt of C10 fatty acid.

The composition is preferably substantially free of octanoic acid or salt of octanoic acid. A C9-C14 fatty acid is a fatty acid having a chain length of 9 carbons to 14 carbons. Octanoic acid can also be called caprylic acid or C8 fatty acid, which is a fatty acid having a chain length of 8 carbons. The present invention also relates to uses of a composition described herein, for example for treating a disease or condition in a subject without adverse effects associated with the stimulation of epithelial-mesenchymal transition (EMT) in the subject, i.e. avoiding or preventing the stimulation of EMT or inhibiting EMT. The present invention also relates to a method for treating a disease or condition without the undesirable stimulation of EMT in a subject in need thereof, comprising administering to the subject a composition described herein, i.e. while preventing or minimizing EMT. Such uses and methods result in no or substantially no increase in EMT, and/or in embodiments may inhibit or reduce EMT, in the subject.

With and without the presence of albumin, the anti-EMT agent disclosed herein have been found to inhibit EMT. Therefore, the present invention also relates to uses of an anti-EMT agent or a combination anti-EMT agents for treating various medical conditions. The present invention also relates to the use of an anti-EMT agent or a combination of anti-EMT agents, for minimizing or reducing epithelial-mesenchymal transition (EMT) in a subject. A composition for use in preventing, inhibiting, minimizing or reducing epithelial-mesenchymal transition (EMT) in a subject, comprising an anti-EMT agent or a combination of anti-EMT agents, is also encompassed.

The salt of the present invention is preferably a pharmaceutically acceptable salt.

### Epithelial-Mesenchymal Transition (EMT) and Endothelial-Mesenchymal Transition (EndoMT)

Epithelial-mesenchymal transition (EMT), and the reverse process, MET (mesenchymal-epithelial transition), are critical processes for development of many tissues and organs during embryogenesis. Recently, endothelial to mesenchymal transition (EndoMT), a newly recognized type of cellular transdifferentiation, has emerged as another possible source of tissue myofibroblasts. EndoMT is a complex biological process in which endothelial cells lose their specific markers and acquire a mesenchymal phenotype and express mesenchymal cell products such as a smooth muscle actin (α-SMA) and type I collagen. Similar to EMT, EndoMT can be induced by transforming growth factor (TGF-β). For the purpose of simplifying the text of the present invention, the term “EMT” is used herein and intends to encompass both EMT and EndoEMT processes which identical processes of cellular transformation/differentiation.

Epithelial (or endothelial) and mesenchymal cells differ from each other with regard to cellular phenotype and function. Epithelial and endothelial cells are closely attached to each other by tight junctions, gap junctions and adherent junctions, exhibit cellular polarity (apico-basal polarity, polarization of the cytoskeleton) and are bound by a basal lamina. Whereas mesenchymal cells lack this cellular polarization, possess a spindle-shaped morphology and minimally interact with each other. Also, epithelial and endothelial cells express characteristic cell markers such as E-cadherin and β-catenin while mesenchymal cells express cell markers such as N-cadherin, fibronectin, vimentin and α-SMA (smooth muscle actin). Additionally, mesenchymal cells possess an increased ability to migrate, compared to epithelial cells. Thus, the process of EMT results in a profound change in cell morphology and phenotype. Broadly speaking, EMT occurs during three processes: embryogenesis, cell proliferation migration, and wound healing. As inhibition of EMT is desirable in certain pathological conditions such as cancer or excessive wound healing (scarring), research has been undertaken regarding the discovery of potential drugs for the inhibition of EMT. Since EMT is characteristic of cells undergoing proliferation, it is induced by protein growth factors such as TGF (Transforming Growth Factor), CTGF (Connective Tissue Growth Factor), EGF (Epidermal Growth Factor), HGF (Hepatocyte Growth Factor) and IGF (Insulin Growth Factor). Subsequently, inhibitors of growth factors offer potential as inhibitors of EMT. For example, the small molecule (MW=384) substituted imidazolylbenzamide SB-431542 is an inhibitor of TGF-β1 receptor (activin receptor-like kinase; Inman et al. (2002), Molecular Pharmacol. 62 (1) 65-74) which attenuates the tumor promoting effects of TGF-β, including TGF-β induced EMT. Similarly, another small molecule EW-7203 blocks TGF-β1-mediated EMT in mammary epithelial cells (Park et al. (2011), Cancer Sci. 102(1):1889-96). Inhibition of an essential kinase (PAK: p21-activated serine/threonine kinase) was also found to inhibit the EMT process (see for example U.S. patent application 2009/0286850).

It has been found that C8 fatty acid or salt thereof increases EMT, whereas any of C9-C14 fatty acid, salt thereof, monoglyceride thereof, diglyceride thereof, and triglyceride thereof decrease EMT. Therefore, in order to prevent the EMT stimulation by the administration of an albumin composition, its content in C8 fatty acid or salt thereof is advantageously lowered or completely avoided. Alternatively, it has been found that replacing a portion of at least 30% of the C8 fatty acid or salt thereof in an albumin composition with an anti-EMT agent will compensate the negative impact of C8 fatty acid or salt thereof. Alternatively, the addition of an anti-EMT agent in an albumin composition so that the anti-EMT agent corresponds to at least 30% of the total content of pro-EMT agent and anti-EMT agent is also contemplated by the present invention.

Also provided is a method of preventing, inhibiting, minimizing or reducing epithelial-mesenchymal transition (EMT) in a subject in need thereof, said method comprising administering to the subject an albumin composition comprising no pro-EMT agent or a low amount of pro-EMT agent; and/or comprising an amount of anti-EMT agent that corresponds to at least 30% of the total amount of pro-EMT and anti-EMT agents.

Also provided is a use of a composition comprising albumin for preventing, inhibiting, minimizing or reducing epithelial-mesenchymal transition (EMT) in a subject in need thereof, wherein said albumin composition comprises no pro-EMT agent or a low amount of pro-EMT agent; and/or comprises an amount of anti-EMT agent that corresponds to at least 30% of the total amount of pro-EMT agent and anti-EMT agent.

Also provided is a use of a composition comprising albumin for the preparation of a medicament for preventing, inhibiting, minimizing or reducing epithelial-mesenchymal transition (EMT) in a subject in need thereof, wherein said albumin composition comprises no pro-EMT agent or a low amount of pro-EMT agent; and/or comprises an amount of anti-EMT agent that corresponds to at least 30% of the total amount of pro-EMT agent and anti-EMT agent.

Also provided is a composition comprising albumin for use in preventing, inhibiting, minimizing or reducing epithelial-mesenchymal transition (EMT) in a subject in need thereof, wherein said albumin composition comprises no pro-EMT agent or a low amount of pro-EMT agent; and/or comprises an amount of anti-EMT agent that corresponds to at least 30% of the total amount of pro-EMT agent and anti-EMT agent.

It is intended that the uses and methods described herein are for preventing, minimizing, reducing, inhibiting, reducing the stimulation of, minimizing the stimulation of, for not stimulating, or for avoiding the stimulation of epithelial-mesenchymal transition (EMT). The present invention also relates to an albumin composition that is devoid of pro-EMT agent and anti-EMT agent, since it will not stimulate EMT. In an alternative embodiment of the invention, an anti-EMT agent is present in the albumin composition. In an alternative embodiment of the invention, a pro-EMT agent is absent from the albumin composition. In an alternative embodiment of the invention, a pro-EMT agent is present at a “low concentration” in the albumin composition, wherein said “low concentration” is below the concentration that is generally used in commercial albumin compositions (i.e. about 0.08 millimole of octoanoate salt per gram of albumin). Preferably, the low concentration of pro-EMT agent is between 0.0007 and 0.07 millimole per gram of albumin, or between 0.0007 and 0.007 millimole per gram of albumin, or between 0.007 and 0.07 millimole per gram of albumin, or between 0.005 and 0.05 millimole per gram of albumin, or between 0.0005 and 0.05 millimole per gram of albumin, or between 0.002 and 0.02 millimole per gram of albumin, between 0.0002 and 0.02 millimole per gram of albumin, between 0.04 and 0.08 millimole per gram of albumin, between 0.04 and 0.07 millimole per gram of albumin, between 0.05 and 0.07 millimole per gram of albumin As described herein a “low concentration” and a “low amount” are two expressions that designates the same since the low amount is relative to the amount of albumin in the composition. In an alternative embodiment, the albumin composition is not devoid of any pro-EMT agent. In another embodiment, the pro-EMT agent is present in the composition of albumin from a concentration of 0.008 millimole per gram of albumin to 0.007% (w/w). In another embodiment, is excluded from the present invention the use of an albumin composition for the purpose of detoxifying a subject, or the use of an albumin composition for the purpose of a detoxification therapy, or a subject in need of detoxification. Albumin composition for detoxifying a subject is disclosed in U.S. Pat. No. 8,877,711. In an embodiment, the subject is in need of EMT prevention or reduction of EMT stimulation, or prevention of excessive scarring, or reduction of the stimulation of excessive scarring, or prevention of cell differentiation, or reduction of the stimulation of cell differentiation.

The present invention further relates to the uses of (i) said albumin composition comprising either no pro-EMT agent or a low amount of pro-EMT agent; and/or comprising an amount of anti-EMT agent that corresponds to at least 30% of the total amount of pro-EMT agent and anti-EMT agent in the albumin composition, and (ii) said composition of anti-EMT agent, such as for therapeutic uses thereof in the treatment of a EMT-related disease or an EMT-related condition, or a disease or condition where the EMT stimulation is not desirable or suitable and/or where the expression of collagen is not desirable or suitable, such as hemorrhage, hypovolemia, burn, acute liver failure, liver dysfunction, hypoalbuminemia, adult respiratory distress syndrome, cirrhosis, neonatal hemolytic disease, cardiopulmonary bypass surgery, nephrosis, cancer, hepatorenal syndrome, sepsis, organ perfusion, organ reperfusion, scar formation, psoriasis and eczema; and in combination with poorly water soluble drugs for the treatment of disease or condition where the EMT stimulation is not suitable or desirable, such as an anticancer drugs. The present invention also relates to the use of a composition of albumin according to the present invention for the preparation of a medicament for treating a disease or a condition for which the administration of an albumin composition is recommended by a physician. The treatment of a disease or a condition for which the administration of an albumin composition is recommended by a physician, includes, but not limited to, hypovolemia, with or without shock; hypoalbuminemia (e.g., due to malnutrition, burns, major injury, congenital analbuminemia, liver disease, liver dysfunction, infection, malignancy, chemotherapy, endocrine disorders, or else), excessive catabolism (e.g., due to burns, major injury, pancreatitis, thyrotoxicosis, pemphigus, nephrosis, or else), loss of albumin from the body (e.g., due to hemorrhage, excessive renal excretion, burn exudates, exudative enteropathy, exfoliative dermatoses, or else) and/or redistribution of albumin within the body (e.g., due to major surgery, orthopedic surgery, cirrhosis with ascites, peritonitis, adhesion, diverticulitis, various inflammatory conditions, or else); prior to or during cardiopulmonary bypass surgery, burns, cirrhosis, albumin dialysis in patients with acute liver failure or acute decompensation of chronic liver disease (e.g., extracorporeal liver support), poisoning, infections, surgical complications, septic shock, severe sepsis. The present invention also relates to the use of a composition of albumin according to the present invention for drug formulation so as to stabilize a drug, or to render a drug more soluble, or to increase the efficacy of a drug or for other purposes, or for delivery vehicle of a drug. In this context, the terms “drug” and “or an active agent” can be interchanged. The present invention also relates to the use of a composition of albumin according to the present invention for cryopreservation of stem cells or embryos for stem cell therapy or in vitro fertilization (IVF).

In an embodiment, the concentration of all C8-14 fatty acid, salt thereof, monoglyceride thereof, diglyceride thereof or triglyceride thereof that is present in the compositions of the present invention is higher or equal or less than 0.08 millimole per gram of albumin. In an embodiment, the total amount of C8-14 fatty acid, salt thereof, monoglyceride thereof, diglyceride thereof or triglyceride thereof is comprised of at least 30% of anti-EMT agent.

The present invention further relates to the uses of an albumin composition comprising an amount of C8-14 fatty acid, salt thereof, monoglyceride thereof, diglyceride thereof or triglyceride thereof that is higher or equal or less than 0.08 millimole per gram of albumin, wherein the amount of pro-EMT agent corresponds to a low concentration with respect to the amount of albumin present in the composition or is absent, and wherein at least 30%, or at least 40%, or at least 50%, or at least 60% of said amount of C8-14 fatty acid, salt thereof, monoglyceride thereof, diglyceride thereof or triglyceride thereof is an anti-EMT agent or a combination thereof. Said uses includes for heat stabilization of serum albumin. It is also encompassed for the present invention that an albumin composition is modified after pasteurization so as to reduce or deplete the content in pro-EMT agent and/or add a content of anti-EMT agent in accordance with the claimed concentration and/or proportion.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

In the studies described herein, the present inventors have demonstrated that the octanoic acid and salt thereof, either in free form or complexed to albumin, stimulates epithelial-mesenchymal transition (EMT), as assessed by an increase in TGF-β-induced collagen expression (a marker of EMT) on human proximal tubule epithelial cells. In contrast, a decrease in TGF-β-induced collagen expression by human proximal tubule epithelial cells (i.e. indicative of an inhibition of EMT) was detected following treatment with other saturated fatty acids (i.e., C9-C14 fatty acids) or triglycerides thereof, either in free form and/or complexed to albumin. Thus, the results presented herein provide evidence that the use of octanoic acid-stabilized HSA may be associated with undesired biological effects, particularly in certain pathological conditions such as cancer or excessive wound healing (scarring) in which stimulation of EMT should be avoided/minimized, and that the use of C9-C14 fatty acid-stabilized HSA, which has an inhibitory effect on EMT, is preferable.

The present invention also relates to a method of preventing, minimizing or reducing the stimulation of epithelial-mesenchymal transition (EMT) in a subject in need thereof, said method comprising administering to the subject a composition that does not contain albumin and comprises:
no pro-EMT agent or a low concentration of pro-EMT agent; and/or
a content of pro-EMT agent and anti-EMT agent in a ratio of pro-EMT agent: anti-EMT agent that is from 7:3 to 0:10.

The pro-EMT agent is as above-described for the albumin composition, i.e. octanoic acid, octanoate salt or a combination thereof. The anti-EMT agent is as above-described for the albumin composition, i.e. a C9-C14 fatty acid, a salt of C9-C14 fatty acid, a monoglyceride of C9-C14 fatty acid, a diglyceride of C9-C14 fatty acid, a triglyceride of C9-C14 fatty acid, or a combination thereof. The concentration of pro-EMT agent is advantageously not too elevated and preferably does exceed 0.02 M. In embodiments, the concentration of pro-EMT agent is 0.01 M or less, 0.001 M or less, 0.0001 M or less. In these embodiments where the composition does not contain albumin, the pro-EMT agent and the anti-EMT agent, when they are fatty acids, they can be separated or distinct compounds or they are part of a diglyceride or a triglyceride while conserving the same ratio. An example of this embodiment is given in Table 2 where a tested triglyceride comprises 2 chains of octanoic acid and one chain of decanoic acid, providing a ratio of 2:1.

The present invention also encompasses the use of a composition for the preparation of a medicament for inhibiting or reducing epithelial-mesenchymal transition (EMT) in a subject, wherein the composition comprises an effective amount of anti-EMT agent as above described. In an embodiment, the effective amount of anti-EMT agent is comprised between 5 mg/kg to 300 mg/kg of the subject, or between 5 mg/kg to 200 mg/kg, or between 5 mg/kg to 100 mg/kg, or between 10 mg/kg to 100 mg/kg per dose for oral or topical administration in a human. In an embodiment, the effective amount of anti-EMT agent is comprised between 0.5 mg/kg to 100 mg/kg, or between 0.5 mg/kg to 50 mg/kg, or between 0.5 mg/kg to 25 mg/kg, or between 0.5 mg/kg to 10 mg/kg, or between 0.5 mg/kg to 4 mg/kg of the subject per dose for intravenous, intraperitoneal, rectal, intramuscular or subcutaneous administration in a human. Said dose is administered once or on a repeated manner during a period of treatment. In an embodiment, said repeated manner is daily, every-two-days, every-three-days, twice-a-week or weekly. Said period of treatment is preferably defined by a physician or until the desired medical results are achieved.

The effective amount can be administered once or on a repeated basis. Repeated administration is from two to four times a day, once a day, every-two-day, every-three-day, twice a week, or once a week.

In an embodiment, the anti-EMT agent is present in a pharmaceutical composition comprising a pharmaceutically acceptable vehicle or carrier.

The term “fatty acid” as used herein refers to a carboxylic acid with an aliphatic chain (either saturated or unsaturated), generally of 4 to 28 carbons. The term “C9-C14 fatty acid” refers to a fatty acid with an aliphatic chain of 9, 10, 11, 12, 13 or 14 carbons, or any mixture thereof. In an embodiment, the aliphatic chain is saturated. The term “triglyceride of C9-C14 fatty acid” refers to a glycerol molecule ester-linked to three C9-C14 fatty acids and may be represented by the following formula:

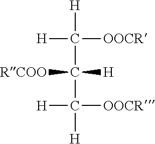

where R′, R″ and R′″ are independently selected from saturated fatty acid or salt having 9, 10, 11, 12, 13 or 14 carbons in the carbon backbone esterified to the glycerol backbone. The term “monoglyceride of C9-C14 fatty acid” refers to a glycerol molecule ester-linked to one C9-C14 fatty acid; and the term “diglyceride of C9-C14 fatty acid” refers to a glycerol molecule ester-linked to two C9-C14 fatty acids. The fatty acid, salt thereof and triglyceride of fatty acid may be prepared by any process known in the art, such as direct esterification, rearrangement, fractionation, transesterification, or the like. For example, the lipids may be isolated from, or derived from, a source of vegetable oil, such as coconut oil, such as through a rearrangement process or the like. The length and distribution of the chain length may vary depending on the source oil. Commercial sources for the C9-C14 fatty acids, salts thereof and triglycerides of C9-C14 fatty acid are available and known to the skilled artisan.

In an embodiment, the albumin composition comprises a single fatty acid of 9, 10, 11, 12, 13 or 14 carbons, or a salt thereof. In another embodiment, the albumin composition comprises any mixture of two or more fatty acids of 9, 10, 11, 12, 13 or 14 carbons, and/or salts thereof.

In embodiments, at least about 40%, 50%, 60%, 70%, 75%, 80%, 85%, 90%, 95%, 96%, 97%, 98% or 99% of the fatty acid and/or salt of fatty acid comprised in the albumin composition is a C9-C14 fatty acid and/or salt thereof. In an embodiment, 100% of the fatty acid and/or salt of fatty acid comprised in the albumin composition is a C9-C14 fatty acid and/or salt thereof.

In embodiments, 60%, 50%, 40%, 30%, 25%, 20%, 15%, 10% or 5% or less of the fatty acid and/or salt of fatty acid comprised in the albumin composition is a C8 fatty acid (“octanoic acid” or “caprylic acid”) and/or salt thereof. In an embodiment, the fatty acid comprised in the albumin composition is free or substantially free of octanoic acid and/or octanoate salt.

The terms “octanoic acid” or “caprylic acid” are used interchangeably herein, and refer to a saturated fatty acid comprising 8 carbon atoms. It has the following structure:

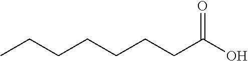

The terms “octanoate salt”, “salt of octanoic acid”, “caprylate salt” and “salt of caprylic acid” are used interchangeably herein.

Both of the expression “reduced amount of pro-EMT agent” and “low concentration of pro-EMT agent” refer to an amount of octanoic acid and/or octanoate salt that is inferior to the amount of octanoic acid and/or octanoate salt that is currently used in commercial albumin preparations on the market. Commercial albumin preparations are using 0.02 M of sodium caprylate in preparation of 25% of albumin, and about 0.004 M in preparation of 5% of albumin. Therefore, the commercially used concentration of sodium caprylate is 0.02 M for 25% of albumin, and it represents 0.08 millimole per gram of albumin. Therefore, a reduced amount of octanoic acid and/or octanoate salt represents less than 0.08 millimole per gram of albumin, or less than 0.05 millimole per gram of albumin, or less than 0.01 millimole per gram of albumin, or 0.005 millimole per gram of albumin or 0.001 millimole per gram of albumin, or less than 0.0005 millimole per gram of albumin, or less than 0.0001 millimole per gram of albumin.

The term “substantially free of octanoic acid and/or octanoate salt” as used herein refers to a concentration of octanoic acid and/or octanoate salt that is not quantifiable or that is not detectable by common techniques. With the techniques known and used by the inventors, the level of quantitation and detection is 0.1 mM, which corresponds to 0.0005 millimole per gram of albumin. Therefore, “substantially free of octanoic acid and/or salt of octanoate salt” represents less than 0.0005 millimole per gram of albumin, or less than 0.0001 millimole per gram of albumin.

The term “free of octanoic acid and/or salt of octanoate salt” as used herein refers to the absence of octanoic acid and/or octanoate salt or the presence of trace thereof. For instance, a composition of albumin that is free of octanoic acid and/or salt thereof is a composition where octanoic acid and/or salt thereof has not been added thereto or a composition where octanoic acid and/or salt thereof has been added and later removed/depleted. For example, a content of octanoic acid and/or salt thereof can be added for stabilization of the albumin during the pasteurization and removed after the pasteurization. For example, the naked albumin of Sigma, which is used in Example 3, is considered as having no fatty acid, although the product specification indicates that it has a trace of fatty acids of 0.007% (w/w) or less. Several methods for fatty acid (pro-EMT agent) removal can be used for the purpose of the present invention, such as (i) ultrafiltration/diafiltration (UF/DF) for exchange with an anti-EMT agent, using a membrane such as hollow fibres, cast membranes, or else; (ii) dialysis with a dialysis tubing for exchange with an anti-EMT agent; (iii) gel filtration, such as a size exclusion column or a desalting column; (iv) adsorption to activated carbon adsorption at low pH following by filtration/purification in order to remove activated carbon; (v) adsorption onto a solid phase, such as carbon particles or various mineral powders, including zeolites, silica, and other; (vi) chromatography; (vii) via hydrophobic column chromatography such as Lipidex 1000 (25 gram with C15 hydrocarbon groups) at 37° C.; (viii) addition of ethanol, optionally pH reduction, followed by heating and filtration to remove unstable precipitated proteins, and UF/DF to displace any residual fatty acids while removing ethanol and/or readjusting pH closer to neutrality; (ix) ion exchange with anion and cation exchange resins and also by dialysis, (x) unfolding albumin to deplete the pro-EMT agent and refolding with the anti-EMT agent (i.e. unfold using a chaotrope (e.g. urea or guanidine hydrochloride) optionally in the presence of a reducing agent (e.g. mercaptoethanol) to unwind the albumin and release bound pro-EMT agent, optionally proceed to a dilution or dialysis/diafiltration to remove any trace of pro-EMT agent, and refold in the presence of anti-EMT agent by removing the presence of chaotrope by dilution, dialysis, diafiltration, or else); or by a combination of these methods.

In an embodiment, at least 30%, or at least 40%, or at least 50%, or at least 60%, or at least 70%, or at least 80%, or at least 85%, or at least 90%, or at least 95%, or 96%, or 97%, or 98%, or 99% or 100% of the content of C8-C14 fatty acid, salt thereof, monoglyceride thereof, diglyceride thereof or triglyceride thereof or a combination thereof (i.e. % of the total content of pro-EMT agent and anti-EMT agent) that is present in the composition is C9-C14 fatty acid, salt of fatty acid, salt thereof, monoglyceride thereof, diglyceride thereof or triglyceride thereof; or preferably C9-C12 fatty acid, salt of fatty acid, salt thereof, monoglyceride thereof, diglyceride thereof or triglyceride thereof, or preferably C10-C12 fatty acid, salt of fatty acid, salt thereof, monoglyceride thereof, diglyceride thereof or triglyceride thereof, or preferably C10 fatty acid, salt of fatty acid, salt thereof, monoglyceride thereof, diglyceride thereof or triglyceride thereof, or preferably decanoic acid and/or decanoate salt. In embodiment, the fatty acid, salt of fatty acid, salt thereof, monoglyceride thereof, diglyceride thereof or triglyceride thereof that is present in the composition is not radiolabelled.

The terms “decanoic acid” or “capric acid” are used interchangeably herein, and refer to a saturated fatty acid comprising 10 carbon atoms. It has the following structure:

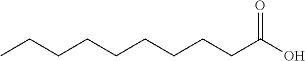

The term “decanoate salt” refers to salt of decanoic acid, salt of capric acid, or salt of caprate.

Advantageously, the salt is pharmaceutically acceptable. Examples of pharmaceutically acceptable salts of C9-C14 fatty acid include sodium, potassium, lithium, ammonium, calcium, magnesium, manganese, zinc, iron, and copper salts; preferably sodium salts, or potassium salts, or a combination of sodium and potassium salts. In an embodiment, the salt is a sodium salt. In an embodiment, the salt C9-C14 fatty acid is a sodium salt, such as sodium decanoate.

In an embodiment, the amount of fatty acid and/or salt thereof, in the albumin composition is an amount that is suitable to stabilize the albumin during the pasteurisation process, which is about 0.02 M for a concentration of 25% of albumin. Lower concentrations of albumin are stabilized with a proportionally lower amount of fatty acid and/or salt thereof. It is contemplated herein that the stabilisation of the albumin can be performed with octanoic acid and/or octanoate salt and that, after the need for stabilisation (i.e. pasteurisation process), octanoic acid and/or octanoate salt is removed from the albumin composition. Such removal is called stripping or depletion. The result of such a removal is an albumin composition free or substantially free of fatty acid or salt thereof, which is also called naked albumin composition. The use of a naked albumin composition is contemplated by the present invention in order to prevent or minimize EMT. Alternatively, the fatty acid or salt thereof used for stabilising albumin is not pure octanoic acid and/or salt thereof and contains at least but not limited to 30%, 50%, 60%, 70%, 75%, 80%, 85%, 90%, 95%, 99%, or 100% of C9-C14 fatty acid and/or salt thereof, or C9-12 fatty acid and/or salt thereof, or C10-C12 fatty acid and/or salt thereof, or C10 fatty acid and/or salt thereof. In these cases where at least 30% of the content of fatty acid and/or salt thereof is C9-C14 fatty acid and/or salt thereof, removal of the fatty acid and/or salt thereof after the need for stabilisation (i.e. pasteurisation process), is not necessary for the purpose of the present invention.

In an embodiment, the amount of albumin in the preparation is from about 5% to less than about 40% (w/v). In an embodiment, the amount of albumin in the preparation is from about 5% to about 25% (w/v). In an embodiment, the amount of albumin in the preparation is from about 20% to about 25% (w/v). In an embodiment, the amount of albumin in the preparation is about 5% (w/v). In an embodiment, the amount of albumin in the preparation is about 20% (w/v). In an embodiment, the amount of albumin in the preparation is about 25% (w/v). The terms “composition of albumin”, “albumin composition” and “albumin preparation” are used interchangeably herein, and means a composition comprising albumin.

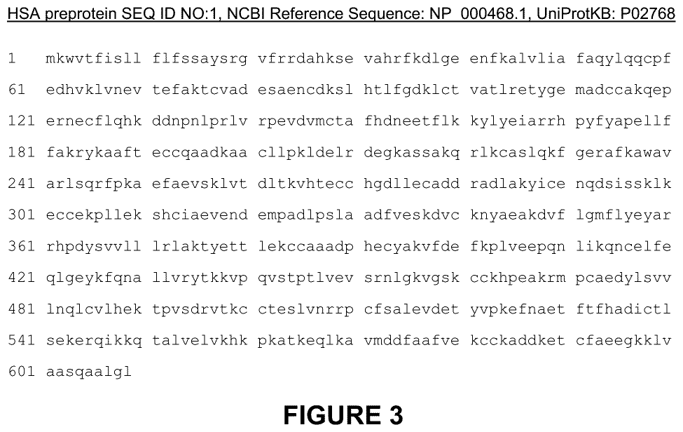

The term “albumin” means a protein having the same and/or very similar tertiary structure as human serum albumin (HSA). In an embodiment, the albumin is native HSA or a variant thereof that retain the functions/properties of HSA. FIG. 3 shows the amino acid sequence of a native HSA preprotein (SEQ ID NO:1, NCBI Reference Sequence: NP_000468.1, UniProtKB: P02768). It should be noted that natural variations of the amino acid sequence of the native HSA preprotein may occur from an individual to another.

The encoded preproprotein is proteolytically processed to generate the mature HSA protein that comprises residues 25-609 of the preproprotein (residues 1-18 define the signal peptide and residues 19-24 define the propeptide).

Some of the major functions/properties of albumin are i) its ability to regulate plasma volume, ii) a long plasma half-life of around 19 days±5 days, iii) ligand-binding, e.g. binding of endogenous molecules such as lipophilic carboxylic acid compounds including bilirubin fatty acids, hemin and thyroxine (see also Table 1 of Kragh-Hansen et al, 2002, Biol. Pharm. Bull. 25, 695), iv) binding of small organic compounds with acidic or electronegative features, e.g. drugs such as warfarin, diazepam, ibuprofen and paclitaxel (see also Table 1 of Kragh-Hansen et al, 2002, supra).

The term “variant” means a polypeptide comprising an alteration, i.e. a substitution, insertion, and/or deletion, at one or more (several) positions relative to native HSA. The altered polypeptide (variant) can be obtained through human intervention by modification of the polynucleotide sequence encoding the native albumin, or can occur naturally. The amino acid sequence of the variant albumin is at least 70%, at least 75%, at least 80%, at least 85%, at least 90%, at least 95%, at least 96%, at least 97%, at least 98%, or at least 99% identical to residues 1-609 of SEQ ID NO:1, and which preferably maintains at least one of the functions/properties of native HSA. For instance, an albumin variant of the present invention includes the fragment of 3-585 sequence. Sequence identity between two amino acid sequences may be determined using well-known algorithms, for example the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al. 2000, Trends Genet. 16: 276-277). Sequence identity may also be determined using the BLAST algorithm, described in Altschul et al., 1990, J. Mol. Biol. 215: 403-10 (using the published default settings). Software for performing BLAST analysis may be available through the National Center for Biotechnology Information.

Thus, in an embodiment, the present invention encompasses albumin composition that comprises an albumin having at least 70% identity with the native mature albumin protein (i.e. residues 25-609 above). In embodiments, the present invention encompasses the use of albumin having at least 75%, 80%, 85%, 90%, 95%, 96%, 97%, 98% or 99% identity with the native mature albumin protein. Such variants of native mature HSA protein may comprise amino acid deletion(s), substitution(s) and/or addition(s) relative to the native mature albumin protein.

The albumin present in the preparation defined herein may be isolated/obtained from any source, for example from blood, plasma, fractionated human serum or through genetic engineering (recombinant albumin). Albumin from fractionated human serum may be prepared for example by the low temperature ethanol fractionation of blood plasma (e.g., the Cohn process and variants thereof). Albumin may also be purified from plasma by chromatography. Albumin may be obtained through a series of affinity and ion exchange columns, applied to plasma and resulting in purified albumin. The purified albumin is typically subjected to thermal treatment at 60° C. for 10 hours, or heat pasteurization, for sterilization. In order to minimize denaturation of albumin due to thermal treatment, the albumin is complexed to fatty acid (or salt thereof). Albumin may also be prepared recombinantly using conventional genetic engineering methods (see, e.g., Sambrook et al. (1989) in Molecular Cloning: A Laboratory Manual; and Sambrook and Russell (2001) Molecular Cloning: A Laboratory Manual (3^{rd} edition; Cold Spring Harbor Laboratory). For example, a recombinant expression vector comprising a nucleic acid sequence encoding an albumin polypeptide may be introduced into a cell, e.g., a host cell (bacterial or eukaryotic cell), which may include a living cell capable of expressing the protein coding region from the defined recombinant expression vector. Vectors can be introduced into cells via conventional transformation or transfection techniques, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), Sambrook and Russell (supra) and other laboratory manuals. The albumin present in the composition of the present invention includes albumin fusion protein and albumin conjugated to a protein through a covalent bond. For instance, fusion may result from albumin that is genetically fused to the C-terminus or the N-terminus of an amino acid sequence (representing a protein or a fragment thereof) via a flexible glycine serine linker or another linker. For instance, conjugation may occur with a protein modified with a maleimide group that reacts and covalently binds to a thiol group of albumin.

HSA variants maintaining at least one of the functions/properties of native HSA are well known in the art. Examples of recombinant serum albumin that may be used herein include those previously disclosed in U.S. Pat. Nos. 5,780,594 and 5,948,609. In addition, a modified or truncated human albumin such as disclosed in U.S. Pat. No. 6,787,636 may also be utilized in the invention. As set forth therein, the serum albumin may be one that has a one- or two-amino acid truncation at its N-terminal end, or any other mutation at the N-terminal end which is sufficient to cause steric hindrance at the N-terminal end so as to reduce the albumin's affinity to trace metals such as copper and/or nickel, thus reducing the likelihood of causing an allergic reaction to the trace metal (i.e. hypoallergenic HSA). Still other forms of HSA may also be suitable for certain applications, such as the albumin variants having altered plasma half-life disclosed in WO2011/051489 and U.S. Pat. No. 8,822,417, WO2011/124718, and WO2012/059486, for example.

In embodiments, the pH of the preparation described herein is about 5 to about 7.5, for example from about 5.5 to about 7.5, preferably from about 6.5 to 7.4.

One or more antioxidants or stabilizers may also be added to the preparation, for example sulfur-containing amino acids, amino acid salts, or derivatives thereof known to exhibit antioxidant or stabilizing activity such as cysteine, cystine, methionine or N-acyl (e.g., N-acetyl) derivatives thereof such as N-acetyl methionine (see U.S. Pat. No. 7,351,800), or other antioxidants or stabilizers such as N-acetyl tryptophan (or N-acetyl tryptophanate). Thus, in an embodiment, the preparation or composition defined herein comprises one or more pharmaceutically acceptable antioxidants or stabilizers, for example the above-noted antioxidants or stabilizers. In a further embodiment, the one or more pharmaceutically acceptable antioxidants or stabilizers comprise N-acetyl methionine and/or N-acetyl tryptophan.

In an embodiment, the one or more pharmaceutically acceptable antioxidants or stabilizers in the preparation is from about 0.001 M to about 0.5 M, from about 0.005 to about 0.1 M, from about 0.01 to about 0.1 M, from about 0.01 to about 0.08 M, or from about 0.01 to about 0.05 M, e.g., about 0.01 M, about 0.02 M, about 0.03 M, about 0.04 M, or about 0.05 M, or a range between any two numerical values above.

In an embodiment, the one or more pharmaceutically acceptable excipients or carriers include, for example, colorants, stabilizers, antiseptics, diluents, pH regulators (e.g., basic amino acid, acidic amino acid, hydrochloric acid, acetic acid, malic acid, or sodium hydroxide), an osmolality regulator (e.g., an electrolyte such as sodium chloride, potassium chloride, potassium gluconate, magnesium sulfate, sodium bicarbonate, calcium chloride, calcium gluconate, or citric acid) and/or a surfactant. In an embodiment, the one or more pharmaceutically acceptable excipients or carriers comprise an osmolality regulator (e.g., electrolytes). In an embodiment, the osmolality regulator is at a concentration of about 10 mM to about 500 mM. In further embodiments, the osmolality regulator is at a concentration of about 50 mM to about 300 mM, about 100 mM to about 200 mM, about 120 mM to about 180 mM or about 125 mM to about 175 mM. In an embodiment, the osmolality regulator comprises sodium, potassium, or both. In a further embodiment, the osmolality regulator comprises sodium and potassium. In an embodiment, the sodium is in an amount of about 130 to about 160 mM. In an embodiment, the potassium is in an amount of about 1 mM to about 5 mM, for example about 2 mM.

The albumin preparations described herein can be prepared by uniformly mixing i) the fatty acid, or salt thereof, or monoglyceride thereof, or diglyceride thereof, or triglyceride thereof or a combination thereof and ii) one or more pharmaceutically acceptable excipients or carriers with iii) an aqueous albumin solution (e.g., a buffer such as phosphate buffer which can be administered as pharmaceutical preparations, injection water, or a physiological saline). After dissolution, the mixture solution is processed into a formulation suitable for administration to a subject, for example for parenteral administration, such as intravenous fluid preparation or an injectable solution. In embodiments, these preparations may be administered subcutaneously or intravenously.

In an embodiment, the albumin preparations and the compositions of anti-EMT agent described herein may be used for the therapeutic indications for which the marketed albumin preparations are currently used for, such as hypovolemia, with or without shock; hypoalbuminemia, which may result from inadequate production of albumin (e.g., due to malnutrition, burns, major injury, congenital analbuminemia, liver disease, infection, malignancy, chemotherapy or endocrine disorders), excessive catabolism (e.g., due to burns, major injury, pancreatitis, thyrotoxicosis, pemphigus, or nephrosis), loss of albumin from the body (e.g., due to hemorrhage, excessive renal excretion, burn exudates, exudative enteropathy, or exfoliative dermatoses) and/or redistribution of albumin within the body (e.g., due to major surgery, orthopedic surgery, cirrhosis with ascites, peritonitis, adhesion, diverticulitis or various inflammatory conditions); prior to or during cardiopulmonary bypass surgery; and for the treatment of burns or cirrhosis. In an embodiment, the albumin preparations or compositions described herein are used for albumin dialysis in patients with acute liver failure or acute decompensation of chronic liver disease (e.g., extracorporeal liver support). In an embodiment, the albumin preparations described herein is used to treat poisoning. In an embodiment, the albumin preparations described herein is used to treat infections, surgical complications, septic shock or severe sepsis. In an embodiment, the albumin preparations described herein is used for drug formulation so as to stabilize a drug, or to render a drug more soluble, or to increase the efficacy of a drug or for other purposes, or for delivery vehicle of a drug. In this context, the terms “drug” and “or an active agent” can be interchanged. In an embodiment, the albumin preparations described herein is used for cryopreservation of stem cells or embryos for stem cell therapy or in vitro fertilization (IVF).

Thus, the present invention provides a method for treating a patient in need for treatment with albumin, said method comprising administering to said subject an effective amount of the albumin preparations or compositions described herein. The present invention provides the use of the albumin preparations described herein for treating a patient in need for treatment with albumin. The present invention provides the albumin preparations described herein for use in treating a patient in need for treatment with albumin.

In an embodiment, said administration or use does not stimulate or increase EMT in said subject. In another embodiment, said administration or use prevents or minimizes EMT or reduces the EMT stimulation in said subject. In an embodiment, such administration or use results in no or substantially no increase in EMT, and in a further embodiment inhibits or reduces EMT, in the subject. As used herein, “does not stimulate or increase EMT”, “prevents or minimizes EMT”, or “results in no or substantially no increase in EMT” means that the administration or uses of a composition or preparation described herein results in no or substantially no increase in EMT compared to commercially known albumin preparations. These expressions also mean that the administration or uses of a composition or preparation described herein results in a lower EMT level in the subject relative to the EMT level resulting from treatment with octanoic acid and/or octanoate salt or a composition comprising albumin and octanoic acid and/or octanoate salt in a concentration of 0.08 millimole per gram of albumin, or in absence of anti-EMT agent. “Inhibits or reduces EMT” means that the administration or uses of a composition or preparation described herein does not only result in no or substantially no increase in EMT, but also reduces the level of EMT in the subject, in comparison with the EMT level of the subject prior to the treatment. “Substantially no increase in EMT” as used herein means that the administration or use results in no significant increase in EMT, which in embodiments refers to less than 10, 9, 8, 7, 6, 5, 4, 3, 2 or 1% increase in EMT, relative to prior to the treatment of the subject with a composition or preparation described herein.

EMT may be assessed by various methods known in the art, for example by assessing the expression or level of one or more markers associated with EMT. Examples include determining the expression or level of an epithelial biomarker and/or the expression or level of an mesenchymal biomarker. Further, numerous reagents, products and kits are commercially available for assessing EMT. In an embodiment, EMT may be determined by assessing TGF-β-induced collagen expression on human proximal tubule epithelial cells, whereby an increase in TGF-β-induced collagen expression on human proximal tubule epithelial cells is indicative of an increase in EMT, and a decrease of TGF-β-induced collagen expression on human proximal tubule epithelial cells is indicative of a decrease in EMT.

In an embodiment, the albumin preparations or compositions of anti-EMT agents described herein are used for the treatment of a disease or condition in which it would be beneficial to prevent or minimize EMT (to prevent or minimize excessive wound healing or scarring), for example in patients suffering from burns (e.g., severe burns or metastasis), patients undergoing organ transplants, patients with hepatorenal disease (e.g., to prevent or reduce organ lesions associated with such conditions), or patients with cancers (e.g. increasing chemotherapy efficacy), or patients with cancers (e.g. preventing the stimulation of EMT that is involved in the metastasis process).

The albumin preparations described herein are generally administered to an adult in a dose of about 5 to 12.5 g of albumin at a time. The albumin preparations or compositions of anti-EMT agent described herein may be administered once a week, twice a week, every-three-days, every-two-days, once a day or about two to four portions a day, or only once, depending on the disease condition.

In an embodiment, albumin in any compositions mentioned herein is in a concentration from 1% to 40%, 2% to 40%, 5% to 40%, from 5% to 30%, from 5% to 25%, from 20% to 25%, of about 5%, of about 10%, of about 15%, of about 20% or of about 25%.

In an embodiment, the albumin preparation or the composition of anti-EMT agent described herein further comprises an active ingredient. In an embodiment, the active ingredient is conjugated to the albumin. In another embodiment, the active ingredient is not conjugated to the albumin.

The term “active agent” can be interchanged with “active ingredient”. The pharmaceutically active agent can be selected from a variety of known classes of drugs, including, for example, analgesics, anesthetics, anti-inflammatory agents, antiparasitic (e.g., anthelmintics), anti-arrhythmic agents, antiasthma agents, antibiotics, anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

In an embodiment, the pharmaceutically active ingredient is insoluble or slightly soluble in water. Examples of pharmaceutically active ingredient that may be incorporated with albumin into the composition include aminoglutethimide, azathioprine, bleomycin sulphate, busulfan, carmustine, chlorambucil, cisplatin, cyclophosphamide, cyclosporine, dacarbazine, dactinomycin, daunorubicin, amycin, etoposide, fluorouracil, interferon-α, lomustine, mercaptopurine, methotrexate, mitotane, procarbazine hydrochloride, thioguanine, vinblastine sulfate, vincristine sulfate, taxanes (e.g., paclitaxel, docetaxel, cabazitaxel, hydrophobic derivatives of docetaxel such as 2′-O-hexanoyldocetaxel, and 2′-benzoyldocetaxel); macrolides such as rapamycin and its derivatives (e.g., temsirolimus and everolimus), epothilone B and its derivatives, tanespimycin and its derivatives; camptothecins, including but not limited to 10-hydroxy camptothecin, SN-38 and its derivatives; anthracycline antibiotics, including but not limited to aclacinomycin and pirarubicin; or other pharmaceutically active ingredients including colchicine and its derivatives, thiocolchicine dimer, amiodardone, liothyronine, cyclosporine, exemestane, flutamide, fulvestrant, romidepsin, semustine, ibuprofen, cyclosporine, propofol, vinblastine, and the like. In some embodiments, the active ingredient is an anti-cancer drug, for example a taxane, camptothecin, irinotecan, gemcitabine, cyclophosphamide (Cytoxan™) doxorubicin, or cisplatin preferably a taxane such as paclitaxel or docetaxel. In particular embodiments, the active ingredient is paclitaxel.

In an embodiment, the albumin is in the form of nanoparticles. Thus, in another aspect, the present invention provides a nanoparticle comprising (i) albumin, (ii) an anti-EMT agent; and (iii) a pharmaceutically active (e.g., therapeutic) agent incorporated or encapsulated within said albumin. Thus, in another aspect, the present invention provides a nanoparticle comprising (i) albumin, (ii) no octanoic acid and/or octanoate salt or a low concentration of octanoic acid and/or octanoate salt. Said “low concentration” is above-described. Nanoparticles comprising albumin and a pharmaceutically active agent, and methods to produce the same, are disclosed for example, in PCT publication No. WO2015/018380 and WO2016/000653.

The term “nanoparticles” as used herein refers to particles having a size in the nano-scale, for example, at the level of about 1 nm, about 10 nm, about 100 nm or about 500 nm. In an embodiment, the nanoparticles have a size of about 1 nm to about 500 nm, about 10 nm to about 200 nm, from example of about 30, 50, 70, 80, or 100 to about 120, 140, 160, 180 or 200 nm, or a range between any two numerical values above.

In an embodiment, the weight ratio of albumin to the pharmaceutically active ingredient in the composition (e.g., nanoparticles) is from about 0.01:1 to about 100:1. In further embodiments, the weight ratio of albumin to the pharmaceutically active ingredient in the composition (nanoparticles) is from about 0.02:1 to about 50:1; from about 0.05:1 to about 20:1; from about 0.1:1 to about 10:1; or from about 0.2:1 to about 5:1, or a range between any two numerical values above.

In an embodiment, the pharmaceutical composition, in liquid form, comprises about 0.1% to about 40% (w/v), e.g. about 0.5% (w/v), 1% (w/v) or 2% (w/v) to about 5% (w/v), about 10% (w/v), about 15% (w/v), about 20% (w/v), about 30% (w/v), or about 35% (w/v) of albumin. In some embodiments, the composition, in liquid form, comprises about 20% to about 25% (w/v) of albumin, or a range between any two numerical values above.

The term “about” as used herein intends to mean +/−10% of the value defined by the term “about” and preferably +/−5% of the value defined by the term “about”.

In an embodiment, the present invention provides a pharmaceutical composition comprising the composition or preparation defined herein and further comprising one or more pharmaceutically acceptable carriers or excipients. Such pharmaceutical compositions may be prepared in a manner well known in the pharmaceutical art. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, rectal, intraperitoneal, intranasal or pulmonary (e.g., aerosol) administration (see Remington: The Science and Practice of Pharmacy, by Loyd V Allen, Jr, 2012, 22^{nd} edition, Pharmaceutical Press Handbook of Pharmaceutical Excipients, by Rowe et al., 2012, 7^{th} edition, Pharmaceutical Press). Therapeutic formulations are prepared using standard methods known in the art by mixing the composition or preparation having the desired degree of purity with one or more optional pharmaceutically acceptable carriers, excipients and/or stabilizers.

An “excipient,” as used herein, has its normal meaning in the art and is any ingredient that is not an active ingredient (drug) itself. Excipients include for example buffers, diluents, lubricants, stabilizing agent, and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. As those of skill would recognize, a single excipient can fulfill more than two functions at once, e.g., can act as both a binding agent and a thickening agent. As those of skill will also recognize, these terms are not necessarily mutually exclusive.

In an embodiment, the albumin preparation or the composition of anti-EMT agent described herein is a suspension or a solution, for example a suspension or a solution for injection. The albumin composition or preparation may be suspended or dissolved in any suitable liquid or diluent, such as a pharmaceutically acceptable or physiologically acceptable liquid which may or may not be or comprise an excipient, carrier or stabilizer. It includes any and all solvents and dispersion media. Examples of liquids or diluents include buffers such as phosphate, citrate, and other organic acids; antioxidants including ascorbic acid. Aqueous liquids or diluents are preferred, such as water (e.g., water for injection), saline such as normal saline 0.9% NaCl (w/v), or an aqueous pH buffered solution.

Formulations for parenteral administration may, for example, contain excipients, sterile water, saline, or polyalkylene glycols such as polyethylene glycol. Biocompatible, biodegradable lactide polymer, lactide/glycolide copolymer, or polyoxyethylene-polyoxypropylene copolymers may be used to control the release of the compounds. Other potentially useful parenteral delivery systems for the albumin preparation or composition of anti-EMT agent described herein include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

The albumin preparation or composition of anti-EMT agent described herein may be in a dry formulation (such as lyophilized composition) or suspended in a biocompatible medium. In embodiments where the pharmaceutical composition is in the form of lyophilized powder, it comprises one or more lyophilization excipients, such as stabilizer(s) and bulking agent(s), mannitol, sucrose, lactose, maltose, trehalose, dextran, buffer, water, or else, or a mixture thereof. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like. In some embodiments, the composition is in sterile, lyophilized powder. In some embodiments, the composition is reconstituted with sterile or water or a buffer. For example, the composition (such as pharmaceutical composition) can be reconstituted in a sodium chloride solution, such as a 0.9% sodium chloride buffer.

In another embodiment, the albumin preparation or composition of anti-EMT agent described herein are for topical administration. The form of the topical preparation can be any form such as a cream, an emulsion, an oil, a foam, a gel, a lotion, an ointment, a paste, a spray or a suspension. The topical preparation/composition optionally comprises one or more topically acceptable auxiliary agents that include, for example, carriers, excipients, emulsifiers, surfactants, preservatives, oils, thickeners, polymers, gel formers, consistency regulators, antioxidants, antifoams, antistats, resins, solvents, solubility promoters, neutralizing agents, stabilizers, sterilizing agents, propellants, water-soluble or dispersible silicone-containing polymers, humectants, emollients or any mixtures thereof. Conventional gelling agents that may be incorporated into topical formulations include one or more of the following, but are not limited to, hydroxyethylcelluose, carbomer, a polyethylene homopolymer, a polyethylene/vinyl acetate copolymer, a polyethylene/acrylic acid copolymer, azelaic acid, aloe vera, lecithin, thermoreversible polysaccarides, and cetylhydroxyethyl cellulose.

If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, coloring agents may be added as well.

In another aspect, the present invention provides methods for using the albumin preparation or composition of anti-EMT agent described herein. The albumin preparation or composition of anti-EMT agent described herein may be used for the treatment of any diseases or disorders that are responsive to the active ingredient.

For example, the albumin preparation or composition of anti-EMT agent described herein may comprise one or more anticancer agents, and may be used in treating cancer, such as liver cancer, prostatic cancer and lung cancer. Additional diseases or disorders that may be treated include breast cancer, multiple myeloma, transplant rejection, colon cancer or lymphoma. In an embodiment, the cancer is not pancreatic cancer.

In a particular aspect, the present invention provides a method for treating cancer that comprises administering a therapeutically effective amount of a pharmaceutical composition or nanoparticles provided herein to a subject in need thereof, wherein the pharmaceutically active ingredient is an anti-tumor or chemotherapeutic agent.

In specific embodiments, the subject is a mammal, including but not limited to human, canine, mouse, and rat. In specific embodiments, the albumin is syngeneic with the subject.

Any suitable amount of the pharmaceutical composition may be administered to a subject. The dosages will depend on many factors including the mode of administration.

For the prevention, treatment or reduction in the severity of a given disease or condition, the appropriate dosage of the composition will depend on the type of disease or condition to be treated, the severity and course of the disease or condition, whether the composition is administered for preventive or therapeutic purposes, previous therapy, the patient's clinical history and response to the composition, and the discretion of the attending physician. The composition is suitably administered to the patient at one time or over a series of treatments. Preferably, it is desirable to determine the dose-response curve in vitro, and then in useful animal models prior to testing in humans. The present invention provides dosages for the compounds and compositions comprising same. For repeated administrations over several days or longer, depending on the condition, the treatment is sustained until a desired suppression of disease symptoms occurs. However, other dosage regimens may be useful. The progress of this therapy is easily monitored by conventional techniques and assays.

### EXAMPLES

Embodiments of the present invention are exemplified herein below.

### Example 1: Preparation of a Composition of Albumin and Sodium Decanoate by Depletion/Replacement of Sodium Octanoate

Human serum albumin (HSA) at a concentration of 200 g/L in a solution of 0.12M NaCl, 30 mM caprylate (sodium octanoate) was used as the starting material (Sigma, Catalog #C5038-1 KG). This sample was diluted 1:20 in 0.12 M NaCl, resulting in an albumin solution at a concentration of about 10 g/L. This sample was then diafiltrated (i.e. buffer exchanged) 8 times with 0.12 M NaCl and re-concentrated to about 50 g/L albumin. A second diafiltration step was then performed with a solution comprising 30 mM sodium decanoate and 0.12 M NaCl for 3 diafiltration volumes, and the sample was brought to a final albumin concentration of 200 g/L with 30 mM sodium decanoate.

Quantitative determination of sodium decanoate in the human serum albumin composition was performed as per the method described in Example 2.

### Example 2: Detection of Sodium Decanoate Vs. Sodium Octanoate in an Albumin Solution

The quantitative method for the determination of sodium octanoate in the albumin solution was based on the publication of Dengler T. et al. Infusionstherapie 15: 273-274 (6/1988). The internal standard for the reversed-phase was sodium decanoate. For the quantitative detection of sodium decanoate, sodium octanoate was used as the standard, and sodium decanoate was the complexing agent. For determination of the relative response at 214 nm and the relative recovery of octanoic and decanoic acid extracted from human serum albumin composition

The albumin composition resulting from the replacement of sodium octanoate with sodium decanoate, and solutions of 40 mM sodium caprylate (sodium octanoate) and 40 mM sodium decanoate, were submitted to HPLC analysis to confirm that sodium octanoate was completely replaced by sodium decanoate in the albumin composition.

Sample preparation: 250 μl of the tested compositions were added to 800 μl of methanol and vortexed for 20 seconds. The precipitate was centrifuged at 10K RPM for 5 minutes and the supernatant was removed and filtered through 0.45 μm Millex™ HV filter. Aliquots were transferred to autosampler vials. Reversed phase high performance liquid chromatography of the extracts was done with a 4.6×250 mm Nucleosil-C18 (5 μm) column with 0.1% trifluoroacetic acid (TFA) in methanol/water (4:1) as mobile phase at a flow rate of 0.8 ml/minute. 20 μl was injected. Detection was done by ultraviolet (UV) absorption at 214 nm. Each sample extract was injected three times and the peak area were averaged. HPLC analysis was done on the Hewlett Packard® Model 1100 HPLC with G1315B diode array detector, G1311A quaternary pump, G1329A thermostatted autosampler, G1316A thermostatted column compartment, and HP Compaq® d530c computer (WIN XP®) with Chemstation® 10.02 software. Performed isocratic reversed phase analysis with Alltech Nucleosil C-18, 4.6 mm×250 mm column, 5 μm.

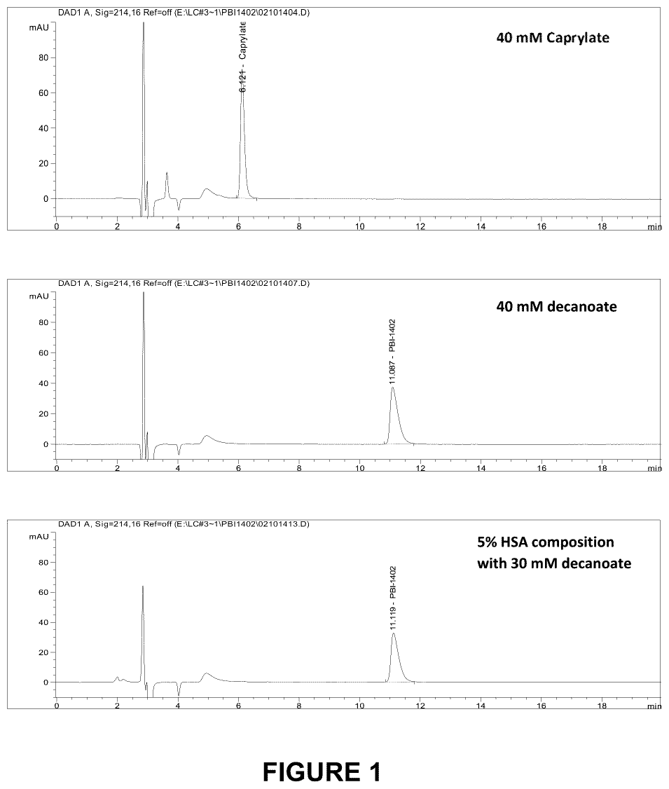

As shown in FIG. 1, it was found that no detectable amount of sodium octanoate remains in the albumin composition prepared in Example 1. This means that the sodium octanoate initially complexed with albumin was successfully removed and replaced with sodium decanoate.

### Example 3: Preparation of an Albumin Composition Comprising Sodium Decanoate and Naked Human Albumin

Human serum albumin (HSA—free fatty acid) from Sigma-Aldrich® (#A1887) was used for this experiment. This human serum albumin is also called “naked human albumin”, it only contains trace of fatty acids of 0.007% (w/w) or less. HSA was dissolved in PBS to obtain a concentration of 6.6% (6.6 g/100 ml), and was filtered using a 0.2 μm filter. 75 μl of sodium octanoate or sodium decanoate 10 mM was mixed with 75 μl of HSA 6.6% (without vortex), and incubated 10 min at room temperature. 100 μL of this mixture was used in the assay of Example 4 in a final volume of 1 ml (dilution 1:10).

### Example 4: EMT Modulation in TGF-β Induced Epithelial Cells (HK-2 Cells)

Analysis was undertaken to determine the effect of various compositions on TGF-β-induced collagen 1 expression, which is a marker of EMT, on immortalized human proximal tubule epithelial cells (HK-2 cells). HK-2 cells were treated with TGF-β (an inducer of EMT) at a concentration of 10 ng/ml. Expression of the EMT marker collagen 1 mRNA was determined by quantitative real-time PCR.

Albumin compositions prepared by the method of Example 1 or Example 3 were formulated with either 2.5 or 5.0×10⁻⁴ M of sodium decanoate or sodium octanoate. Sodium decanoate and sodium octanoate alone (i.e. without albumin) at the same concentrations, as well as nonaic acid, undecanoic acid and sodium laurate at the indicated concentrations, were tested in parallel.

Table 1 shows that sodium decanoate, either alone or formulated with albumin decrease (or inhibit) the expression of collagen in TGF-β-induced HK-2 cells. Nonanoic acid, undecanoic acid and sodium laurate used alone also inhibited the expression of collagen in TGF-β-induced HK-2 cells. In contrast, the expression of collagen was increased (or stimulated) in TGF-β-induced cells treated with sodium octanoate alone or formulated with albumin. Thus, sodium decanoate and sodium octanoate had opposite effects on EMT as assessed by the expression of TGF-β-induced collagen 1 in HK-2 cells, with sodium decanoate inhibiting EMT and sodium octanoate stimulating EMT, and such effects are retained in the presence of albumin. Nonanoic acid, undecanoic acid and sodium laurate appears to exhibit an effect on EMT similar to that of sodium decanoate. The presence and absence of albumin in the composition has no effect on the EMT, evidencing that the inhibition or stimulation of EMT is the result of the fatty acid or salt present in the composition. “HSA” refers to an albumin composition prepared from naked albumin according to Example 3. “ALE” refers to an albumin composition prepared by depletion of sodium octanoate and replacement with sodium decanoate according to Example 1.

TABLE 1
Collagen mRNA expression by HK-2 cells following treatment with
C8-C12 fatty acid or salt thereof; alone or in combination with albumin.

| | Fatty acid | | Changes in collagen |
|---|---|---|---|
| | carbon | Fatty acid | mRNA expression (relative |
| Compound(s) | number | concentration | to TGF-β-treated cells) |
| Sodium | 8 | 5.0 × 10⁻⁴M | 24.1% of increase |
| Octanoate | | 2.5 × 10⁻⁴M | 42.2% of increase |
| HSA/Sodium | 8 | 5.0 × 10⁻⁴M | 58.3% of increase |
| Octanoate | | 2.5 × 10⁻⁴M | 28.7% of increase |
| ALB/Sodium | 8 | 5.0 × 10⁻⁴M | 37.0% of increase |
| Octanoate | | 2.5 × 10⁻⁴M | 28.7% of increase |
| Nonanoic Acid | 9 | 6.3 × 10⁻⁷M | 51.4% of decrease |
| Decanoic Acid | 10 | 5.8 × 10⁻⁷M | 105.4% of decrease |
| Sodium | 10 | 5.0 × 10⁻⁴M | 103.4% of decrease |
| Decanoate | | 2.5 × 10⁻⁴M | 66.6% of decrease |
| HSA/Sodium | 10 | 5.0 × 10⁻⁴M | 54.1% of decrease |
| Decanoate | | 2.5 × 10⁻⁴M | 19.1% of decrease |
| ALB/Sodium | 10 | 5.0 × 10⁻⁴M | 86.7% of decrease |
| Decanoate | | 2.5 × 10⁻⁴M | 71.7% of decrease |
| Undecanoic Acid | 11 | 2.7 × 10⁻⁷M | 120.95% of decrease |
| Sodium Laurate | 12 | 1.0 × 10⁻⁴M | 113.7% of decrease |

The effect of free C10-C14 triglycerides (with saturated carbon chains) on collagen mRNA expression by HK-2 cells was also tested. The results are reported in Table 2.

TABLE 2
Collagen mRNA expression by HK-2 cells following treatment with
compositions comprising C10-C14 triglycerides

| | | | Changes in collagen |
|---|---|---|---|
| | | | mRNA expression |
| Triglyceride | Carbon number | Triglyceride | (relative to TGF-β-treated |
| name | in the chains | concentration | cells) |
| Tricaprin | C10 | 5% | 120.4% of decrease |
| Triglyceride | C8 and C10 | 5% | 46.05% of decrease |
| composed of | in 2:1 ratio | | |
| two C8 fatty | | | |
| acids and one | | | |
| C10 fatty acid | | | |
| Trilaurin | C12 | 5% | 84.6% of decrease |
| Trimyristin | C14 | 5% | 52.3% of decrease |

By comparing the results in Table 2 for tricaprin and the triglyceride mixture having 70% of 08 chain and 30% of 010 chain, it can be noted that the presence of 70% of 08 chain reduces the inhibition of collagen expression.

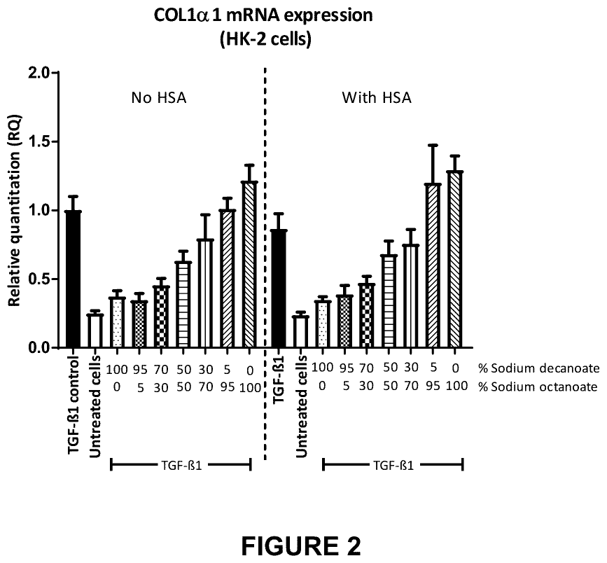

Ratios of sodium octanoate: sodium decanoate of 100:0, 95:5, 70:30, 50:50, 30:70, 5:95 and 0:100 have been tested in compositions with albumin and without albumin, in the in vitro model above-described. The various combinations of salt of fatty acid and albumin have been prepared by the method described in Example 3. The collagen 1a1 mRNA expression is reported in FIG. 2. Real-Time PCR was using human TaqMan Gene Expression Assay normalized to human GAPDH endogenous control. Reference is TGF-β1 control (RQ=1). The * and ** mean a p value of 0.05 and 0.01 respectively, compared to reference. Tested concentration of the mixture of sodium octanoate and sodium decanoate is 500 μM. The number of experiments is 3 (n=3).

Firstly, the results shown in FIG. 2, confirm that albumin has no impact on collagen expression, and therefore, no impact on EMT. Secondly, it can be noted that the expression of collagen is dose-dependent of the proportion of sodium decanoate versus sodium octanoate. Thirdly, it can be noted that a significant reduction of collagen expression is observable when sodium decanoate is present at 30% of the total salt of fatty acid content or greater. Ratios of 70:30, 50:50, 30:70, 5:95 and 0:100 of sodium octanoate: sodium decanoate significantly reduce or inhibit the collagen expression. This confirms that the ratio of 70:30 of sodium octanoate: sodium decanoate is the minimal ratio where the inhibition effect of sodium decanoate successfully compensates for the stimulation effect of sodium octanoate. The stimulation effect of sodium octanoate is evidenced in this study in ratios of 100:0 and 95:5 sodium octanoate: sodium decanoate where the observed collagen expression is raised above the level of the control (TGF-β1 treated HK-2 cells).

### Example 5: EMT Modulation in TGF-β Induced Human Microvascular Endothelial Cells (HMEC)

Endothelial to mesenchymal transition (EndoMT), which is included in the definition of the expression EMT as used in the present description, can be induced by transforming growth factor (TGF-β). Analysis of EndoMT was undertaken, and the method of analysis is described in Example 4. Real-Time PCR was using human TaqMan Gene Expression Assay normalized to human GAPDH endogenous control. Reference is TGF-β1 control (RQ=1). The ** and *** mean a p value of 0.01 and 0.001 respectively, compared to reference. Tested concentration of sodium octanoate and sodium decanoate is 500 μM. The number of experiments is 3 (n=3).

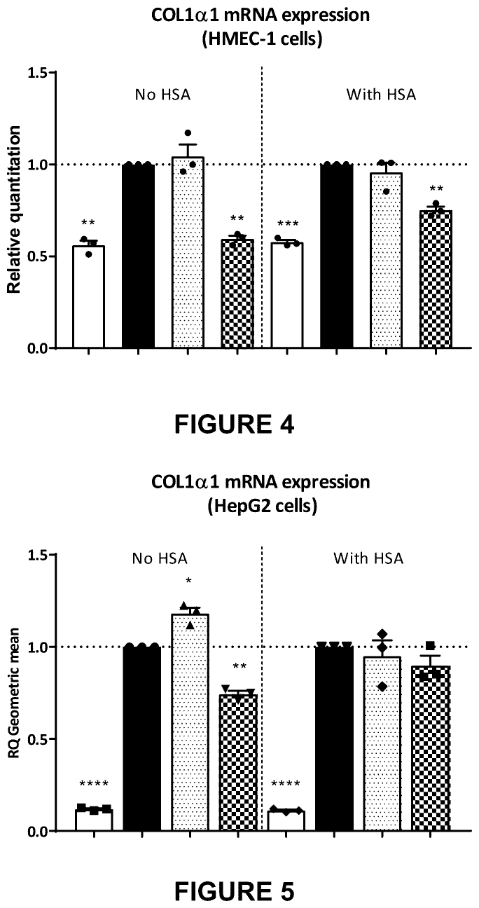

FIG. 4 shows the induction of EndoMT by TGF-β, and is expressed by the increase in collagen 1a1 mRNA expression. FIG. 4 also shows that sodium decanoate, both in presence of albumin (With HSA) or absence of albumin (No HSA), has reduced significantly the mesenchymal transition that has been induced by TGF-β, contrarily to sodium octanoate. Therefore, the anti-EMT agent of the present invention successfully inhibits or reduces EMT in endothelial cells.

### Example 6: EMT Modulation in TGF-β Induced Human Hepatocellular Carcinoma Cells: HepG2

Analysis of EMT was undertaken, following the method that is described in Example 4, in human hepatocellular carcinoma cell line HepG2. These cells are highly differentiated and display many of the genotypic features of normal liver cells (Sassa et al. 1987). Consequently, HepG2 cells can be used to screen for EMT and as illustrated in FIG. 5, TGF-β also increased collagen 1a1 mRNA expression, a marker of EMT. Real-Time PCR was using human TaqMan Gene Expression Assay normalized to human GAPDH endogenous control. Reference is TGF-β1 control (RQ=1). The *, ** and **** mean a p value of 0.05, 0.01 and 0.0001 respectively, compared to reference. Tested concentration of sodium octanoate and sodium decanoate is 500 μM. The number of experiments is 3 (n=3).

FIG. 5 shows that sodium decanoate, both in presence of albumin (With HSA) or absence of albumin (No HSA), has reduced significantly the mesenchymal transition that has been induced by TGF-β, contrarily to sodium octanoate. Therefore, the anti-EMT agent of the present invention successfully inhibits or reduces EMT in liver cells.

### Example 7: EMT Modulation in TGF-β Induced Human Lung Epithelial Cell A549

Analysis of EMT was also undertaken by the method described in Example 4, in human lung epithelial cell A549. TGF-β also increased collagen 1a1 mRNA expression in human lung epithelial cell A549, a marker of EMT. Real-Time PCR was using human TaqMan Gene Expression Assay normalized to human GAPDH endogenous control. Reference is TGF-β1 control (RQ=1). The *** and **** mean a p value of 0.001 and 0.0001 respectively, compared to reference. Tested concentration of sodium octanoate and sodium decanoate is 500 μM. The number of experiments is 3 (n=3).

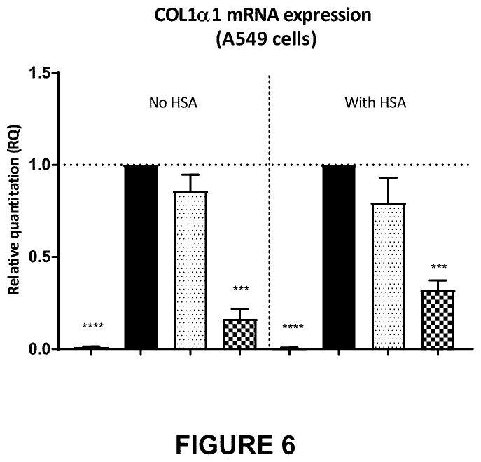

FIG. 6 shows that sodium decanoate, both in presence of albumin (With HSA) or absence of albumin (No HSA), has reduced significantly the mesenchymal transition that has been induced by TGF-β, contrarily to sodium octanoate which has provided no significant results. Therefore, the anti-EMT agent of the present invention successfully inhibits or reduces EMT in lung cells.

### Example 8: EMT Modulation in TGF-β Induced Human Prostate Cancer PC-3

The epithelial-mesenchymal transition (EMT) is a process by which epithelial cells lose their cell polarity and cell-cell adhesion, and gain migratory and invasive properties to become mesenchymal stem cells; these are multipotent stromal cells that can differentiate into a variety of cell types. EMT is essential for numerous developmental processes including mesoderm formation and neural tube formation. EMT has also been shown to occur in wound healing, in scarring process and in the initiation of metastasis in cancer progression.

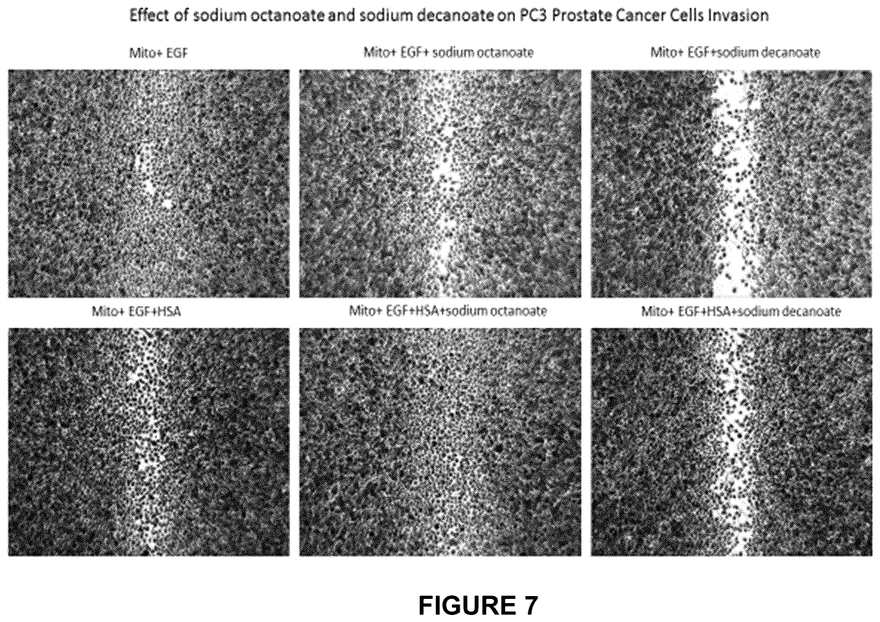

The present experiment exemplifies EMT analysis of cancer cells through invasion/migration of PC-3 cell in a “scratch assay”. This assay is based on the observation that, upon creation of a new artificial gap, so called ‘scratch’, on a confluent cell monolayer, the cells on the edge of the newly created gap will move toward the opening to close the scratch. EGF (epithelial growth factor) was used to induce the migratory/invasion of PC-3 cells into the scratch. Briefly, culture-inserts are placed in a 24-well cell culture plate. PC-3 cells are seeded in culture-inserts and are incubated overnight at 37° C. in a CO₂ incubator. Cells are then starved with serum-free medium for another 24 hours. The insert is removed, leaving a visible 500 μm cell-free gap. Cells were treated for 24 h with EGF at 50 ng/mL and Mitomycin C at 10 μg/mL (Mito+EGF) and with a pro-EMT agent, sodium octanoate (Mito+EGF+sodium octanoate) or an anti-EMT agent, sodium decanoate (Mito+EGF+sodium decanoate), with HSA (second row in FIG. 7) and without HSA (first row in FIG. 7). Pictures are taken at 1 h post-treatment as a confluence control for each group. After 24 h, cells are fixed with Crystal Violet staining and air dried before 24 h post treatment pictures are taken. Pictures are reported in FIG. 7.

It can be noted in FIG. 7 that EGF promotes the migration or invasion of PC-3 cells treated with mitomycin. FIG. 7 demonstrates that the addition of sodium decanoate (with and without albumin) to the cell culture produces an inhibition of the EGF-induced PC-3 migration or invasion, whereas sodium octanoate does not inhibit the cell migration. This migration is increased when sodium octanoate is combined with albumin.

The scope of the claims should not be limited by the preferred embodiments set forth in the examples, but should be given the broadest interpretation consistent with the description as a whole.

1.-36. (canceled)

37. A composition for treating cancer in a subject, comprising
an anticancer drug;
a C8-fatty acid or salt thereof; and
a C9-C12 fatty acid or salt thereof,
wherein the C9-C12 fatty acid or salt thereof is present in an amount of at least 30% of the total amount of the C8-fatty acid or salt thereof and the C9-C12 fatty acid or salt thereof, and
wherein the C8 fatty acid or salt thereof and the C9-C12 fatty acid or salt thereof are present as free acid or monoglyceride, diglyceride, or triglyceride thereof.

38. The composition of claim 37, wherein the C9-C12 fatty acid or salt thereof is present in an amount in a range of 40% to 95% of the total amount of the C8 fatty acid or salt thereof and the C9-C12 fatty acid or salt thereof.

39. The composition of claim 37, wherein the C9-C12 fatty acid or salt thereof is C10 fatty acid or salt thereof.

40. The composition of claim 39, wherein the C10 fatty acid or salt thereof is present in an amount in a range of 5 mg/kg to 300 mg/kg of the subject per dose.

41. The composition of claim 39, wherein the C10 fatty acid or salt thereof is present in an amount in a range of 0.5 mg/kg to 4 mg/kg per dose.

42. The composition of claim 39, wherein the C10 fatty acid or salt thereof is present in an amount of about 70% of the total amount of the C8-fatty acid or salt thereof and the C10 fatty acid or salt thereof.

43. The composition of claim 37, wherein the anticancer drug is a taxane.

44. The composition of claim 43, wherein the taxane is paclitaxel.

45. The composition of claim 37, wherein the composition is an injectable composition.

46. The composition of claim 37 further comprising albumin.

47. The composition of claim 39, wherein the C10 fatty acid and the C8 fatty acid are present on the same triglyceride.

48. The composition of claim 37, wherein the cancer is prostate cancer, lung cancer, or breast cancer.

49. A composition for treating prostate cancer, lung cancer, or breast cancer in a subject, comprising
paclitaxel;
a C8-fatty acid; and
a C10 fatty acid,
wherein the C10 fatty acid is present in an amount of at least 30% of the total amount of the C8-fatty acid and the C10 fatty acid,
wherein the C8 fatty acid and the C10 fatty acid are present as free acid or monoglyceride, diglyceride, or triglyceride thereof.

50. The composition of claim 49, wherein the C10 fatty acid is present in an amount in a range of about 40% to about 95% of the total amount of the C8-fatty acid and the C10 fatty acid.

51. The composition of claim 49, wherein the C10 fatty acid is present in an amount of about 70% of the total amount of the C8-fatty acid and the C10 fatty acid.

52. The composition of claim 49, wherein the C10 fatty acid and the C8 fatty acid are present on the same triglyceride.

53. The composition of claim 49 further comprising albumin.

54. The composition of claim 49, wherein the composition is an injectable composition.
